SEQUENCE LISTING (18 sequences)

SEQ ID NO: 1 - DNA, 1008 bp, Thermoascus aurantiacus
  sig_peptide (1)..(90)
  CDS (1)..(1005)
  mat_peptide (91)..(1005)
atgaagctcggctctctcgtgctcgctctcagcgcagctaggcttacactgtcggcccctctcgcagacagaaagcaggagaccaagcgtgcgaaagtattccaatggttcggttcgaacgagtccggtgctgaattcggaagccagaaccttccaggagtcgagggaaaggattatatatggcctgatcccaacaccattgacacattgatcagcaaggggatgaacatctttcgtgtcccctttatgatggagagattggttcccaactcaatgaccggctctccggatccgaactacctggcagatctcatagcgactgtaaatgcaatcacccagaaaggtgcctacgccgtcgtcgatcctcataactacggcagatactacaattctataatctcgagcccttccgatttccagaccttctggaaaacggtcgcctcacagtttgcttcgaatccactggtcatcttcgacactaataacgaataccacgatatggaccagaccttagtcctcaatctcaaccaggccgctatcgacggcatccgttccgccggagccacttcccagtacatctttgtcgagggcaattcgtggaccggggcatggacctggacgaacgtgaacgataacatgaaaagcctgaccgacccatctgacaagatcatatacgagatgcaccagtacctggactctgacggatccgggacatcagcgacctgcgtatcttcgaccatcggtcaagagcgaatcaccagcgcaacgcagtggctcagggccaacgggaagaagggcatcatcggcgagtttgcgggcggagccaacgacgtctgcgagacggccatcacgggcatgctggactacatggcccagaacacagacgtctggactggcgccatctggtgggcggccgggccgtggtggggagactacatattctccatggagccggacaatggcatcgcgtatcagcagatacttcctattttgactccgtatctttga

SEQ ID NO: 2 - protein, 335 aa, Thermoascus aurantiacus
MKLGSLVLALSAARLTLSAPLADRKQETKRAKVFQWFGSNESGAEFGSQNLPGVEGKDYIWPDPNTIDTLISKGMNIFRVPFMMERLVPNSMTGSPDPNYLADLIATVNAITQKGAYAVVDPHNYGRYYNSIISSPSDFQTFWKTVASQFASNPLVIFDTNNEYHDMDQTLVLNLNQAAIDGIRSAGATSQYIFVEGNSWTGAWTWTNVNDNMKSLTDPSDKIIYEMHQYLDSDGSGTSATCVSSTIGQERITSATQWLRANGKKGIIGEFAGGANDVCETAITGMLDYMAQNTDVWTGAIWWAAGPWWGDYIFSMEPDNGIAYQQILPILTPYL

SEQ ID NO: 3 - protein, 21 aa, Thermoascus aurantiacus
  MISC_FEATURE: N-terminal peptide
NXLVFTSFGSNESGAEFGSQN

SEQ ID NO: 4 - DNA, 20 bp, Artificial
  Primer
aaygartcnggngcngaatt

SEQ ID NO: 5 - DNA, 20 bp, Artificial
  Primer
aaygartcnggngcngagtt

SEQ ID NO: 6 - DNA, 20 bp, Artificial
  Primer
aaygaragkggngcngaatt

SEQ ID NO: 7 - DNA, 20 bp, Artificial
  Primer
aaygaragkggngcngagtt

SEQ ID NO: 8 - DNA, 18 bp, Artificial
  Primer
aagatgtactgggaagtg

SEQ ID NO: 9 - DNA, 21 bp, Artificial
  Primer
tggttgagattgaggactaag

SEQ ID NO: 10 - DNA, 21 bp, Artificial
  Primer
gattatagaattgtagtatct

SEQ ID NO: 11 - DNA, 19 bp, Artificial
  Primer
agagccggtcattgagttg

SEQ ID NO: 12 - DNA, 20 bp, Artificial
  Primer
atgaagctcggctctctcgt

SEQ ID NO: 13 - DNA, 21 bp, Artificial
  Primer
cttgtctcctgtctcgttcac

SEQ ID NO: 14 - protein, 225 aa, Thermomyces lanuginosus
  mat_peptide (31)..(225)
MVGFTPVALAALAATGALAFPAGNATELEKRQTTPNSEGWHDGYYYSWWSDGGAQATYTNLEGGTYEISWGDGGNLVGGKGWNPGLNARAIHFEGVYQPNGNSYLAVYGWTRNPLVEYYIVENFGTYDPSSGATDLGTVECDGSIYRLGKTTRVNAPSIDGTQTFDQYWSVRQDKRTSGTVQTGCHFDAWARAGLNVNGDHYYQIVATEGYFSSGYARITVADVG

SEQ ID NO: 15 - protein, 439 aa, Peniophora lycii
  mat_peptide (31)..(439)
MVSSAPAPSILLSLMSSLALSTGPSPVAAGLPIPAGATSATGPTAPPPPVGPTAAPPGGCTVTGVALIGAHGAATPTSGAASAGVAAVALIGMAAPPTAPLTGPLAAPVTLPGVAALLPPGAAGSHGTGTAMTTATSTLPGGGAVPPVAAAGAGAVVASSTATTAGPGAASGGTVLPTLGVVLGGGGACTLCAAMCPAGVAGAGSTTTLGVPAPAITAALAAAAPSAALSASAALTLMAMCPPATLSSGAASPPCALPTAGGTVSTGTTTALALTTGTGPGAALGPVGGVGTVAGLLAALTGGAVAAGTGTAATLASAPATPPLAATPTAAPSHAATMVPIPAALGLPAATALAPLLPAGAALTVASLLVPPSGHMTVGLLACSGLGAVAVLVAAAVGPLGPCGGVAGVCGLSAPVGSGTTAAGAGGGAPALCGPVPSG

SEQ ID NO: 16 - protein, 332 aa, Myceliophthora thermophila
  mat_peptide (1)..()
ALTYRGVDWSSVVVEERAGVSYKNTNGNAQPLENILAANGVNTVRQRVWVNPADGNYNLDYNIAIAKRAKAAGLGVYIDFHYSDTWADPAHQTMPAGWPSDIDNLSWKLYNYTLDAANKLQNAGIQPTIVSIGNEIRAGLLWPTGRTENWANIARLLHSAAWGIKDSSLSPKPKIMIHLDNGWDWGTQNWWYTNVLKQGTLELSDFDMMGVSFYPFYSSSATLSALKSSLDNMAKTWNKEIAVVETNWPISCPNPRYSFPSDVKNIPFSPEGQTTFITNVANIVSSVSRGVGLFYWEPAWIHNANLGSSCADNTMFSQSGQALSSLSVFQRI

SEQ ID NO: 17 - DNA, 1008 bp, Thermoascus aurantiacus; identical to SEQ ID NO: 1
  CDS (1)..(1005)
  sig_peptide (1)..(96)
  mat_peptide (97)..(1005)

SEQ ID NO: 18 - protein, 335 aa, Thermoascus aurantiacus; identical to SEQ ID NO: 2

(19) United States
(12) Patent Application Publication
(10) Pub. No.: US 2006/0193843 A1
(43) Pub. Date: Aug. 31, 2006

(54) Thermostable enzyme compositions

(75) Inventors: Wenping Wu, Beijing (CN); Dan Pettersson, Lynge (DK); Claus Crone Fuglsang, Veksoe (DK)

Correspondence Address: NOVOZYMES NORTH AMERICA, INC., 500 FIFTH AVENUE, SUITE 1600, NEW YORK, NY, 10110, US

(73) Assignee: DSM IP ASSETS B.V., Heerlen (NL)

(21) Appl. No.: 10/500,477

(22) PCT Filed: Jan. 23, 2003
(86) PCT No.: PCT/DK03/00039

§ 371 Date: Jan. 21, 2005

(30) Foreign Application Priority Data
Jan. 25, 2002 (DK) PA 2002 00130

(51) Int. Cl. A61K 38/43

(52) U.S. Cl. 424/94.1

(57) ABSTRACT

The present invention relates to a composition comprising at least two thermostable enzymes selected from the group consisting of endoglucanase, xylanase, phytase, protease, galactanase, mannanase, dextranase, and alpha-galactosidase. The thermostable enzymes have a melting temperature, Tm, of at least 70° C. Preferred compositions comprise a xylanase of glycoside hydrolase family 11, and an endoglucanase which is homologous to a thermostable glycoside hydrolase family 5 endoglucanase derived from Thermoascus aurantiacus. Preferred xylanases are derived from Aspergillus, Bacillus, Humicola, Thermomyces and Trichoderma. The composition is particularly useful for animal feed purposes. Optional additional components are vitamins, minerals, and anti-microbial peptides.

### FIELD OF THE INVENTION

The present invention relates to compositions comprising at least two thermostable enzymes selected from the group consisting of: Endoglucanase, xylanase, phytase, protease, galactanase, mannanase, dextranase, and alpha-galactosidase; as well as the preparation and use thereof, in particular in relation to animal feed.

### BACKGROUND OF THE INVENTION

A thermostable xylanase derived from Thermomyces lanuginosus (SEQ ID NO: 14) is disclosed in WO 96/23062. The amino acid sequence of an endo-beta-1,4-glucanase derived from Thermoascus aurantiacus IFO 9748 was submitted to NCBI Entrez Protein Database (accession no. GenPept AAL 16412.1) on 10 Sep. 2001. Examples of thermostable phytases are the various consensus phytases listed in WO 99/48380 at p. 30, below the bold line.

### SUMMARY OF THE INVENTION

The present invention relates to compositions comprising at least two thermostable enzymes selected from the group consisting of: Endoglucanase, xylanase, phytase, protease, galactanase, mannanase, dextranase, and alpha-galactosidase. The invention also relates to methods of preparing such compositions, their use in animal feed, their use for treatment of vegetable proteins, as well as animal feed compositions with content thereof.

### DETAILED DESCRIPTION OF THE INVENTION

While enzymes in solid form may to a certain extent be protected from being damaged by heat by way of protective coatings and the like, there is a need, in particular for animal feed purposes, for liquid enzymes of an inherently high thermostability (per se thermostable enzymes).

Many animal feed enzyme preparations are multicomponent enzyme preparations obtained by submerged fermentation of various microorganisms. However, a number of monocomponent feed enzymes prepared by recombinant DNA technology are also available. Monocomponent feed enzymes may have certain advantages as compared to the traditional multicomponent enzyme preparations.

The present invention provides improved enzyme compositions, in particular of relevance within the field of animal feed.

In the present context, the expressions “enzyme” and “polypeptide having enzyme activity” are used interchangeably.

### Thermostable Enzymes

For the present purposes, the term thermostable means that the polypeptide has a melting temperature, Tm, using Differential Scanning Calorimetry (DSC) of at least 70° C., as determined at a pH in the interval of 5.0 to 7.0. In particular embodiments, the Tm is at least 71, 72, 73, 74, 75, 76, 77, 77.5, 78, 79, 80, 81, 82, 83, 84, 85, 86, 87, 88, 89, 90, 91, 92, 93, 94, 95, 96, 97, 98, 99 or at least 100° C. In alternative embodiments, the Tm is at least 64, 65, 66, 67, 68, or at least 69° C.

For the determination of Tm, an enzyme sample with a purity of at least 90% (or 91, 92, 93, 94, 95, 96, 97, or 98%) as determined by SDS-PAGE may be used. Still further, the enzyme sample may have a concentration of between 0.5 and 2.5 mg/ml protein (or between 0.6 and 2.4, or between 0.7 and 2.2, or between 0.8 and 2.0 mg/ml protein), as determined from absorbance at 280 nm and based on an extinction coefficient calculated from the amino acid sequence of the enzyme in question.

The DSC may take place at any pH value in the interval of pH 5.0-7.0, for instance at pH7.0 (e.g. in a buffer of 10 mM phosphate, 50 mM NaCl), or at pH 6.5, 6.0, 5.5 or 5.0; and with a constant heating rate, e.g. of 1, 1.5, 2, 3, 4, 5, 6, 7, 8, 9 or 10° C./min. Examples of preferred heating rates are 1.0, 1.5 or 2.0° C./min when using an equipment as described in Example 6 herein. For other types of equipment with smaller sample volumes Tm can be estimated using a heating rate of, e.g., 3, 4, 5, 6, 7, 8, 9 or 10° C./min, or a heating rate of 20, 30, 40, 50 or even up to 60°C./min.

### Enzyme Compositions

The composition of the invention comprises at least two enzymes selected from thermostable endoglucanases, xylanases, phytases, proteases, galactanases, mannanases, dextranases, and alpha-galactosidases.

In particular embodiments, the composition comprises thermostable enzymes belonging to two, three, four, five, six, seven or all eight of these classes of enzymes. More than one enzyme of each class may be included, e.g. one, two, three, four, etc.

Particular compositions of the invention comprise at least two thermostable enzymes selected from the group consisting of endoglucanase, xylanase, phytase and galactanase. Examples of such compositions are: Endoglucanase and xylanase; endoglucanase and phytase; endoglucanase and galactanase; xylanase and phytase; xylanase and galactanase; phytase and galactanase; endoglucanase, xylanase and phytase; endoglucanase, xylanase and galactanase; endoglucanase, phytase and galactanase; xylanase, phytase and galactanase; endoglucanase, xylanase, phytase and galactanase. In a preferred embodiment, these compositions are combined with at least one protease, mannanase, dextranase and/or alpha-galactosidase.

Further particular compositions of the invention comprise at least two thermostable enzymes selected from the group consisting of endoglucanase, xylanase, phytase, protease and galactanase.

Additional particular compositions of the invention comprise at least two thermostable enzymes selected from the group consisting of endoglucanase, xylanase and phytase. Examples of such compositions are: Endoglucanase and xylanase; endoglucanase and phytase; xylanase and phytase; endoglucanase, xylanase and phytase. In a preferred embodiment, these compositions are combined with at least one galactanase, protease, mannanase, dextranase and/or alpha-galactosidase.

Still further particular compositions of the invention comprise the following thermostable enzymes: Endoglucanase and xylanase; endoglucanase and protease; endoglucanase, xylanase and phytase; endoglucanase, xylanase and protease; endoglucanase, xylanase, phytase and protease; xylanase and phytase; xylanase and protease; phytase and protease; phytase, protease and galactanase; xylanase, phytase and protease; xylanase, protease and galactanase; phytase and galactanase; galactanase and protease; phytase, galactanase and alpha-galactosidase; phytase and alpha-galactosidase; protease and alpha-galactosidase; galactanase and alpha-galactosidase; galactanase, protease and alpha-galactosidase.

Of these compositions, the following are particularly useful feed additives for (a) maize and soy bean based diets: phytase and protease; phytase, protease and galactanase; (b) wheat and soy bean based diets: xylanase and protease; galactanase, protease and xylanase; (c) wheat-barley and soy bean based diets: xylanase, betaglucanase and protease; xylanase, betaglucanase and phytase; for barley and soy bean based diets: beta-glucanase and protease.

### EC Classes of Enzymes—Bernard Henrissat Glycoside Hydrolase Families

Enzymes can be classified on the basis of the handbook Enzyme Nomenclature from NC-IUBMB, 1992), see also the ENZYME site at the internet: http://www.expasy.ch/enzyme/. ENZYME is a repository of information relative to the nomenclature of enzymes. It is primarily based on the recommendations of the Nomenclature Committee of the International Union of Biochemistry and Molecular Biology (IUB-MB) and it describes each type of characterized enzyme for which an EC (Enzyme Commission) number has been provided (Bairoch A. The ENZYME database, 2000, Nucleic Acids Res 28:304-305). This IUB-MB Enzyme nomenclature is based on their substrate specificity and occasionally on their molecular mechanism; such a classification does not reflect the structural features of these enzymes.

Another classification of certain glycoside hydrolase enzymes, such as endoglucanase, xylanase, galactanase, mannanase, dextranase and alpha-galactosidase, in families based on amino acid sequence similarities has been proposed a few years ago. They currently fall into 90 different families: See the CAZy(ModO) internet site (Coutinho, P. M. & Henrissat, B. (1999) Carbohydrate-Active Enzymes server at URL: http://afmb.cnrs-mrs.fr/˜cazy/CAZY/index.html (corresponding papers: Coutinho, P. M. & Henrissat, B. (1999) Carbohydrate-active enzymes: an integrated database approach. In “Recent Advances in Carbohydrate Bioengineering”, H. J. Gilbert, G. Davies, B. Henrissat and B. Svensson eds., The Royal Society of Chemistry, Cambridge, pp. 3-12; Coutinho, P. M. & Henrissat, B. (1999) The modular structure of cellulases and other carbohydrate-active enzymes: an integrated database approach. In “Genetics, Biochemistry and Ecology of Cellulose Degradation”, K. Ohmiya, K. Hayashi, K. Sakka, Y. Kobayashi, S. Karita, and T. Kimura eds., Uni Publishers Co., Tokyo, pp. 15-23).

### Polypeptides having Xylanase Activity

For the present purposes a xylanase is an enzyme classified as EC 3.2.1.8 (see the ENZYME site referred to above). The official name is endo-1,4-beta-xylanase. The systematic name is 1,4-beta-D-xylan xylanohydrolase. Other names may be used, such as endo-(1-4)-beta-xylanase; (1-4)-beta-xylan 4-xylanohydrolase; endo-1,4-xylanase; xylanase; beta-1,4-xylanase; endo-1,4-xylanase; endo-beta-1,4-xylanase; endo-1,4-beta-D-xylanase; 1,4-beta-xylan xylanohydrolase; beta-xylanase; beta-1,4-xylan xylanohydrolase; endo-1,4-beta-xylanase; beta-D-xylanase. The reaction catalysed is the endohydrolysis of 1,4-beta-D-xylosidic linkages in xylans.

According to the CAZy(ModO) site referred to above, xylanases are presently classified in either of the following Glycoside Hydrolyase Families: 10, 11, 43, 5 or 8.

Family 11 glycoside hydrolases can be characterized as follows:
CAZy Family: Glycoside Hydrolase Family 11
Known Activities: Xylanase (EC 3.2.1.8)
Mechanism: Retaining
Catalytic Nucleophile/Base: Glu (experimental)
Catalytic Proton Donor: Glu (experimental)
3D Structure Status: Available (see PDB).
Fold: Beta-jelly roll
Clan: GH-C

In particular embodiments, the thermostable xylanase of the composition of the invention is i) a xylanase of Glycoside Hydrolyase Family 10, 11, 43, 5 or 8; ii) a xylanase of i) with the exception of Thermoascus aurantiacus xylanase, iii) a xylanase of i) with the exception of the xylanase designated xyna_theau and described in J. Mol. Biol. (1999) 288, 999-1012 by Natesh et al; iv) a xylanase of Glycoside Hydrolase Family 11, 43, 4 or 8; or v) a xylanase of Glycoside Hydrolase Family 11. The expression “of Glycoside Hydrolase Family NN” means that the xylanase in question is or can be classified in family “NN” (e.g. 10, 11, 43, 5or 8).

In another particular embodiment, the thermostable xylanase is derived from a bacterial xylanase, e.g. a Bacillus xylanase, for example from a strain of Bacillus halodurans, Bacillus pumilus, Bacillus agaradhaerens, Bacillus circulans, Bacillus polymyxa, Bacillus sp., Bacillus stearothermophilus, or Bacillus subtilis, including each of the Bacillus xylanase sequences entered at the CAZy(ModO) site referred to above.

In a further particular embodiment the family 11 glycoside hydrolase is a fungal xylanase. Fungal xylanases include yeast and filamentous fungal polypeptides as defined above, with the proviso that these polypeptides have xylanase activity.

Examples of fungal xylanases of family 11 glycoside hydrolase are those which can be derived from the following fungal genera: Aspergillus, Aureobasidium, Emericella, Fusarium, Gaeumannomyces, Humicola, Lentinula, Magnaporthe, Neocallimastix, Nocardiopsis, Orpinomyces, Paecilomyces, Penicillium, Pichia, Schizophyllum, Talaromyces, Thermomyces, Trichoderma.

Examples of species of these genera are listed below in the general polypeptide section.

The sequences of xylanase polypeptides deriving from a number of these organisms have been submitted to the databases GenBank/GenPept and SwissProt with accession numbers which are apparent from the CAZy(ModO) site.

A preferred fungal xylanase of family 11 glycoside hydrolases is a xylanase derived from

(i) Aspergillus, such as SwissProt P48824, SwissProt P33557, SwissProt P55329, SwissProt P55330, SwissProt Q12557, SwissProt Q12550, SwissProt Q12549, SwissProt P55328, SwissProt Q12534, SwissProt P87037, SwissProt P55331, SwissProt Q12568, GenPept BAB20794.1, GenPept CAB69366.1;

(ii) Trichoderma, such as SwissProt P48793, SwissProt P36218, SwissProt P36217, GenPept AAG01167.1, GenPept CAB60757.1;

(iii) Thermomyces or Humicola, such as SwissProt Q43097; or

(iv) a xylanase having an amino acid sequence of at least 75% identity to a (mature) amino acid sequence of any of the xylanases of (i)-(iii); or

(v) a xylanase encoded by a nucleic acid sequence which hybridizes under low stringency conditions with a mature xylanase encoding part of a gene corresponding to any of the xylanases of (i)-(iii);

(vi) a variant of any of the xylanases of (i)-(iii) comprising a substitution, deletion, and/or insertion of one or more amino acids;

(vii) an allelic variant of (i)-(iv);

(viii) a fragment of (i), (ii), (iii), (iv) or (vi) that has xylanase activity; or

(ix) a synthetic polypeptide designed on the basis of (i)-(iii) and having xylanase activity.

Definitions, specific conditions, parameters, as well as particular embodiments of these preferred fungal xylanases forming part of the composition of the invention are apparent from the general polypeptide section hereof (just replace “polypeptide” with “xylanase”). This is for example the case for the calculation of percentage identity, and for the selection of hybridization conditions.

A preferred xylanase is the Thermomyces xylanase of SwissProt Q43097 (of which the mature peptide corresponds to amino acids 31-225 of SEQ ID NO: 14), or analogues thereof as defined in (iv)-(ix) above. This xylanase is also described in WO096/23062, and it has a Tm at pH 7.0 of 75.0° C. (see Example 6).

Various Aspergillus xylanases are also described in EP 695349, EP 600865, EP 628080, and EP 532533. EP 579672 describes a Humicola xylanase.

Xylanase activity can be measured using any assay, in which a substrate is employed, that includes 1,4-beta-D-xylosidic endo-linkages in xylans. Assay-pH and assay-temperature are to be adapted to the xylanase in question. Examples of assay-pH-values are pH 4, 5, 6, 7, 8, 9, 10, or 11. Examples of assay-temperatures are 30, 35, 37, 40, 45, 50, 55, 60, 65, 70 or 80° C.

Different types of substrates are available for the determination of xylanase activity e.g. Xylazyme cross-linked arabinoxylan tablets (from MegaZyme), or insoluble powder dispersions and solutions of azo-dyed arabinoxylan.

For assaying xylanase in feed, premix and the like samples, the enzyme is extracted at temperatures ranging from 50° C. up to 70° C. (with the higher temperatures used for the more thermostable enzymes) in an extraction media typically consisting of a phosphate buffer (0.1 M and a pH adjusted to the pH optima of the enzyme in question) for a time period of 30 to 60 min. A preferred xylanase assay is disclosed in Example 7.

All measurements are based on spectrophotometric determination principles at approx. 590-600 nm. The enzyme, or the extracted enzyme, as applicable, is incubated with a known amount of substrate and the colour release is measured relative to a standard curve obtained by adding known amounts of an enzyme standard to a similar control diet without enzyme. When no control feed is available, a known amount of enzyme is added to the sample (spiking) and from the differences in response between spiked and non-spiked sample the added amount of enzyme can be calculated.

### Polypeptides having Endoglucanase Activity

For the present purposes, the term endoglucanase designates any enzyme which is classified or can be classified as EC 3.2.1.4, EC 3.2.1.6, EC 3.2.1.73, or EC 3.2.1.39 (see below under endo-1,3(4)-betaglucanase).

According to the ENZYME site referred to above, endoglucanases are classified as EC 3.2.1.4. The official name is cellulase. Other names may be used, such as endoglucanase, endo-1,4-beta-glucanase, and carboxymethyl cellulase. The reaction catalysed is endohydrolysis of 1,4beta-D-glucosidic linkages in cellulose. Also 1,4-linkages in beta-D-glucans also containing 1,3-linkages will be hydrolysed by such enzyme.

According to the CAZy(ModO) site referred to above, endoglucanases are presently classified in either of the following Glycoside Hydrolyase Families: 10, 12, 26, 44, 45, 5, 51, 6, 61, 7, 74, 89, or not yet assigned to a family.

In particular embodiments, the thermostable endoglucanase of the composition of the invention is i) an enzyme classified as EC 3.2.1.4 or EC 3.2.1.6; ii) an enzyme classified as EC 3.2.1.4; iii) an endoglucanase of Glycoside Hydrolyase Family 10, 12, 26, 44, 45, 5, 51, 6, 61, 7, 74, or 89; or iv) an endoglucanase of Glycoside Hydrolase Family 5. The expression “of Glycoside Hydrolase Family NN” means that the xylanase in question is or can be classified in family “NN” (e.g. 10, 12, 26, 44, 45, 5, 51, 6, 61, 7, 74, or 89).

Family 5 glycoside hydrolases can be characterized as follows:
CAZy Family: Glycoside Hydrolase Family 5
Known Activities: endoglucanase (EC 3.2.1.4); beta-mannanase (EC 3.2.1.78); exo-1,3-glucanase (EC 3.2.1.58); endo-1,6-glucanase (EC 3.2.1.75); xylanase (EC 3.2.1.8); endoglycoceramidase (EC 3.2.1.123)
Mechanism: Retaining
Catalytic Nucleophile/Base: Glu (experimental)
Catalytic Proton Donor: Glu (experimental)
3D Structure Status: Available (see PDB).
Fold: (beta/alpha)₈
Clan: GH-A

Examples of family 5 glycoside hydrolases having endoglucanase activity are apparent from the CAZy(ModO) site. Included is, for example, an endoglucanase derived from Thermoascus aurantiacus IFO 9748 (GenPept AAL 16412.1).

Definitions, specific conditions, parameters, as well as particular embodiments of the endoglucanases forming part of the composition of the invention are apparent from the general polypeptide section hereof (just replace “polypeptide” with “endoglucanase”). This is for example the case for the calculation of percentage identity, and for the selection of hybridization conditions.

In a particular embodiment, the polypeptide is a polypeptide derived from a filamentous fungus of the phylum Ascomycota, preferably of the class Eurotiomycetidae, more preferably of the order Eurotiales, even more preferably of the family Trichocomaceae.

In another embodiment, the polypeptide is derived from a fungus of the genus Thermoascus, for example the species Thermoascus aurantiacus, such as the strain Thermoascus aurantiacus CGMCC No. 0670, e.g., a polypeptide with the amino acid sequence of amino acids 1-335, or 31-335 of SEQ ID NO:2. This endoglucanase (also having endo-1,3(4)-beta-glucanase activity) is thermostable as disclosed in the experimental part hereof (Tm of 77.5° C.).

Endoglucanase activity can be determined using any endoglucanase assay known in the art. For example, various cellulose- or beta-glucan-containing substrates can be applied, under conditions adapted to the enzyme under evaluation (a pH close to the optimum pH and a temperature close to the optimum temperature). A preferred assay pH is in the range of 2-10, preferably 3-9, more preferably pH 3 or 4 or 5 or 6 or 7 or 8, for example pH 3 or pH 7. A preferred assay temperature is in the range of 20-80° C., preferably 30-80° C., more preferably 40-75° C., even more preferably 40-60° C., preferably 40 or 45 or 50° C. The enzyme activity is defined by reference to appropriate blinds, e.g. a buffer blind. These assay conditions are generally applicable for any of the enzymes described herein.

An example of a preferred endoglucanase assay using AZCL-Barley beta-Glucan (AZO-Barley beta-Glucan) as a substrate is described in Examples 3 and 7, respectively. The assay may be modified to use AZCL-HE-Cellulose as a substrate. In both cases, the degradation of the substrate is followed spectrophotometrically at about OD₅₉₅ (see the Megazyme method for AZCL-polysaccharides for the assay of endo-hydrolases (http://www.megazyme.com/booklets/AZCLPOL.pdf). For the purposes of the present invention, endoglucanase activity may also be determined according to the procedure described in Example 1, where the enzyme catalyzes the degradation of an Azo-CM-cellulose substrate using a temperature and a pH at which the actual enzyme is active.

### Polypeptides having Endo-1,3(4)-beta-glucanase Activity

According to the ENZYME site referred to above, endo-1,3(4)-beta-glucanases are usually classified as EC 3.2.1.6. The official name is endo-1,3(4)-beta-glucanase. Other names may be used, such as endo-1,4-beta-glucanase, endo-1,3-beta-glucanase, or laminarinase. The reaction catalysed is endohydrolysis of 1,3- or 1,4-linkages in beta-D-glucans when the glucose residue whose reducing group is involved in the linkage to be hydrolysed is itself substituted at C-3. Substrates for this type of enzyme include laminarin, lichenin and cereal D-glucans.

For the purposes of the present invention, also the following two enzyme classes are included in the term “endo-1,3(4)-beta-glucanase:”

Class EC 3.2.1.73, the official name of which is licheninase. Other names are lichenase, beta-glucanase, endo-beta-1,3-1,4 glucanase, 1,3-1,4-beta-D-glucan 4-glucanohydrolase, or mixed linkage beta-glucanase. The reaction catalysed is hydrolysis of 1,4-beta-D-glycosidic linkages in beta-D-glucans containing 1,3- and 1,4-bonds. This enzyme class acts on lichenin and cereal beta-D-glucans, but not on beta-D-glucans containing only 1,3- or 1,4-bonds.

Class EC 3.2.1.39, the official name of which is glucan endo-1,3-beta-D-glucosidase. Other names are (1-3)-beta-glucan endohydrolase, endo-1,3-beta-glucanase, or laminarinase. The reaction catalysed is hydrolysis of 1,3-beta-D-glucosidic linkages in 1,3-beta-D-glucans. It has a very limited action on mixed-link (1,3-1,4)-beta-D-glucans, but hydrolyses laminarin, paramylon and pachyman.

According to the CAZy(ModO) site referred to above, endo-1,3(4)-beta-glucanases are presently classified in Glycoside Hydrolase Family 16.

Family 16 glycoside hydrolases can be characterized as follows:
CAZy Family: Glycoside Hydrolase Family 16
Known Activities: lichenase (EC 3.2.1.73); xyloglucan xyloglucosyltransferase (EC 2.4.1.207); agarase (EC 3.2.1.81); kappa-carrageenase (EC 3.2.1.83); endo-beta-1,3-glucanase (EC 3.2.1.39); endo-beta-1,3-1,4-glucanase (EC 3.2.1.6); endo-beta-galactosidase (EC 3.2.1.103)
Mechanism: Retaining
Catalytic Nucleophile/Base: Glu (experimental)
Catalytic Proton Donor: Glu (experimental)
3D Structure Status: Available (see PDB).
Fold: Beta-jelly roll
Clan: GH-B

Examples of endo-1,3(4)-beta-glucanases are apparent from the CAZy(ModO) site.

Endo-1,3(4)-beta-glucanases may be derived as described in the general polypeptide section hereof (just replace “polypeptide” with “endoglucanase”).

Endo-1,3(4)-beta-glucanase activity can be determined using any endo-1,3(4)-beta-glucanase assay known in the art. For example, any of the substrates mentioned above can be applied, under conditions adapted to the enzyme under evaluation (e.g. a pH close to the optimum pH and a temperature close to the optimum temperature of the enzyme in question).

A preferred substrate for endo-1,3(4)-beta-glucanase activity measurements is a cross-linked azo-coloured beta-glucan Barley substrate. All measurements are based on spectrophotometric determination principles. For samples of enzyme in feed or premix, the enzyme is extracted at a temperature of 60° C. in a 1/30 M Sorensen buffer (0.24 g Dinatriumhydrogenphosphate-Dihydrat (Merck 6580) and 22.47 g Kaliumdihydrogen-phosphate (Merck 4873), in. 4500 ml deionised water, pH is adjusted to 5.00 with HCl and diluted to 50000 ml final volume) following a general procedure similar to that for xylanase determination except that a control feed always must be used to eliminate the endogenous endo-1,3(4)-beta-glucanase background from barley.

Both methods can also be applied to premixes if the premix to be analysed is mixed with a suitable control feed (as described in connection with the assays of Example 1).

For the purposes of the present invention, the endo-1,3(4)-beta-glucanase activity is preferably determined according to the procedure described in Example 1.

For the purposes of the present invention, the polypeptide having endo-1,3(4)-beta-glucanase activity may be the same as, or different from, the polypeptide having endoglucanase activity.

### Polypeptides having Protease Activity

The term protease as used herein is an enzyme that hydrolyses peptide bonds (has protease activity). Proteases are also called e.g. peptidases, proteinases, peptide hydrolases, or proteolytic enzymes.

Preferred proteases for use according to the invention are of the endo-type that act internally in polypeptide chains (endopeptidases). Endopeptidases show activity on N- and C-terminally blocked peptide substrates that are relevant for the specificity of the protease in question.

Included in the above definition of protease are any enzymes belonging to the EC 3.4 enzyme group (including each of the thirteen sub-subclasses thereof).

Proteases are classified on the basis of their catalytic mechanism into the following groupings, each of which is a particular embodiment of a protease comprised in a composition of the invention: Serine proteases (S), cysteine proteases (C), aspartic proteases (A), metalloproteases (M), and unknown, or as yet unclassified, proteases (U), see Handbook of Proteolytic Enzymes, A. J. Barrett, N. D. Rawlings, J. F. Woessner (eds), Academic Press (1998), in particular the general introduction part.

Protease activity can be measured using any assay, in which a substrate is employed, that includes peptide bonds relevant for the specificity of the protease in question. Assay-pH and assay temperature are to be adapted to the protease in question. Examples of assay-pH-values are pH 3, 4, 5, 6, 7, 8, 9, 10, or 11. Examples of assay temperatures are 25, 30, 35, 37, 40, 45, 50, 55, 60, 65, or 70° C.

Examples of protease substrates are casein, and pNA-substrates, such as Suc-AAPF-pNA (available e.g. from Sigma S-7388). The capital letters in this pNA-substrate refers to the one-letter amino acid code. Another example is Protazyme AK (azurine dyed crosslinked casein prepared as tablets by Megazyme T-PRAK).

Example 2 of WO 01/58276 describes suitable protease assays. A preferred assay is the Protazyme assay of Example 2D (the pH and temperature should be adjusted to the protease in question as generally described previously). For assaying protease in feed or premix, the extraction methods as described herein, e.g. in Example 1 for endoglucanase and xylanase assays, can be used.

In particular embodiments, the protease is a serine protease, a subtilisin protease as defined in WO 01/58275, or a metalloprotease.

Examples of preferred proteases are those described in:
WO 95/02044 (Aspergillus aculeatus protease I or protease II);
JP 407 5586 (Aspergillus niger acid proteinase (protease A));
Berka al, Gene 86:153-162, 1993 (Aspergillus oryzae aspergillopepsin O);
EP 704167 at p. 8, line 51 to p. 9, line 9;
WO 01/58276 at p. 4, line 25 to p. 5, line 18;
WO 01/58275 at p. 5, line 17 to p. 6, line 5;

the section entitled “Summary of the Invention” of pending patent application PCT/DK02/00824 (claiming the priority of DK PA 2001 01821 filed 07.12.01 in the name of Novozymes A/S); or

an analogue, a fragment, a variant, a mutant of any of the above, as described in the general polypeptide part hereof.

Preferred thermostable protease variants have a degree of identity to any one of the proteases listed in WO 01/58276 at p. 4, line 25 to p. 5, line 18; or WO 01/58275 at p. 5, line 17 to p.6, line 5 of at least 75%.

Definitions, specific conditions, parameters, as well as particular embodiments of these preferred proteases forming part of the composition of the invention are apparent from the general polypeptide section hereof (just replace “polypeptide” with “protease”). This is for example the case for the calculation of percentage identity, and for the selection of hybridization conditions.

Polypeptides having Phytase Activity In the present context a phytase is an enzyme which catalyzes the hydrolysis of phytate (myo-inositol hexakisphosphate) to (1) myo-inositol and/or (2) mono-, di-, tri-, tetra- and/or penta-phosphates thereof and (3) inorganic phosphate.

According to the ENZYME site referred to above, two different types of phytases are known: A so-called 3-phytase (myo-inositol hexaphosphate 3-phosphohydrolase, EC 3.1.3.8) and a so-called 6-phytase (myo-inositol hexaphosphate 6-phosphohydrolase, EC 3.1.3.26). For the purposes of the present invention, both types are included in the definition of phytase.

For the purposes of the present invention phytase activity may be, preferably is, determined in the unit of FYT, one FYT being the amount of enzyme that liberates 1 micro-mol inorganic ortho-phosphate per min. under the following conditions: pH 5.5; temperature 37° C.; substrate: sodium phytate (C₆H₆O₂₄P₆Na₁₂) in a concentration of 0.0050 mol/l. Suitable phytase assays are described in Example 1 of WO 00/20569. FTU is for determining phytase activity in feed and premix. In the alternative, the same extraction principles as described in Example 1, e.g. for endoglucanase and xylanase measurements, can be used for determining phytase activity in feed and premix.

Examples of thermostable phytases are disclosed in WO 99/49022 (Phytase variants), WO 99/48380 (Thermostable phytases, see in particular Example 3 thereof), WO 00/43503 (Consensus phytases), EP 0897010 (Modified phytases), EP 0897985 (Consensus phytases).

Thermostable phytases may also be obtained from, e.g., the following phytases:

(i) Ascomycetes, such as those disclosed in EP 684313 or U.S. Pat. No. 6,139,902; Aspergillus awamori PHYA (SWISSPROT P34753, Gene 133:55-62 (1993)); Aspergillus niger (ficuum) PHYA (SWISSPROT P34752, Gene 127:87-94 (1993), EP 420358); Aspergillus awamori PHYB (SWISSPROT P34755, Gene 133:55-62 (1993)); Aspergillus niger PHYB (SWISSPROT P34754, Biochem. Biophys. Res. Commun. 195:53-57(1993)); Emericella nidulans PHYB (SWISSPROT O00093, Biochim. Biophys. Acta 1353:217-223 (1997));

(ii) Thermomyces or Humicola, such as the Thermomyces lanuginosus phytase disclosed in WO 97/35017;

(ii) Basidiomycetes, such as Peniophora (WO 98/28408 and WO 98/28409);

(iii) Other fungal phytases such as those disclosed in JP 11000164 (Penicillium phytase), or WO98/13480 (Monascus anka phytase);

(iv) Bacillus, such as Bacillus subtilis PHYC (SWISSPROT O31097, Appl. Environ. Microbiol. 64:2079-2085 (1998)); Bacillus sp. PHYT (SWISSPROT O066037, FEMS Microbiol. Lett. 162:185-191 (1998); Bacillus subtilis PHYT_(SWISSPROT P42094, J. Bacteriol. 177:6263-6275 (1995)); the phytase disclosed in AU 724094, or WO 97/33976;

(v) Escherichia coli (U.S. Pat. No. 6,110,719);

(vi) Schwanniomyces occidentalis (U.S. Pat. No. 5,830,732);

(vii) a phytase having an amino acid sequence of at least 75% identity to a (mature) amino acid sequence of a phytase of (i)-(vi); or

(viii) a phytase encoded by a nucleic acid sequence which hybridizes under low stringency conditions with a mature phytase encoding part of a gene corresponding to a phytase of (i)-(vi);

(ix) a variant of the phytase of (i)-(vi) comprising a substitution, deletion, and/or insertion of one or more amino acids;

(vii) an allelic variant of (i)-(vi);

(viii) a fragment of (i), (ii), (iii), (iv) or (vi) that has phytase activity; or

(x) a synthetic polypeptide designed on the basis of (i)-(vi) and having phytase activity.

Preferred thermostable phytases for use according to the invention are the various thermostable variants of the Peniophora lycii phytase (mature peptide corresponding to amino acids 31-225 of SEQ ID NO: 15). These thermostable variants are disclosed in DK patent applications no. 2002 00193 and 2002 01449, filed 08.02.2002, and 30.09.2002, respectively. The thermostable variants have a degree of identity to amino acids 31-225 of SEQ ID NO: 15 of at least 75%.

Definitions, specific conditions, parameters, as well as particular embodiments of these preferred phytases forming part of the composition of the invention are apparent from the general polypeptide section hereof (just replace “polypeptide” with “phytase”). This is for example the case for the calculation of percentage identity, and for the selection of hybridization conditions.

### Polypeptides having Galactanase Activity

The term galactanase as used herein is an enzyme that catalyzes the endohydrolysis of 1,4-beta-D-galactosidic linkages in arabinogalactans. The IUBMB Enzyme Nomenclature is EC 3.2.1.89. The official name is arabinogalactan endo-1,4-beta-galactosidase. Alternative Names are endo-1,4-beta-galactanase, galactanase, and arabinogalactanase.

In particular embodiments, the galactanase of the composition of the invention i) is or can be classified as EC 3.2.1.89; and/or ii) is or can be classified as a Glycoside Hydrolase Family 53 galactanase.

GH family 53 is characterized as follows:
Known Activities: Endo-1,4-beta-galactanase (EC 3.2.1.89).
Mechanism: Retaining
Catalytic Nucleophile/Base: Glu (experimental)
Catalytic Proton Donor: Glu (experimental)
3D Structure Status Available (see PDB). Fold (beta/alpha)₈
Clan: GH-A

These are examples of galactanases:

| Protein | Organism | GenBank | GenPept | SwissProt | Publication |
|---|---|---|---|---|---|
| galacta- | Aspergillus aculeatus | L34599 | AAA32692.1 | P48842 | Christgau et |
| nase 1 | | | | | al, Curr. Genet. |
| | | | | | 27: 135- |
| | | | | | 141(1995) |
| endo-1,4- | Aspergillus niger | AJ305303 | CAC83735.1 | Q8X168 | — |
| beta- | | | | | |
| galacta- | | | | | |
| nase | | | | | |
| (GalA) | | | | | |
| galacta- | Aspergillus | AJ012316 | CAB40555.1 | Q9Y7F8 | Van der Vlugt- |
| nase GalA | tubingensis | | | | Bergmans et |
| | | | | | al, Biotechnol. |
| | | | | | Tech. 13:87- |
| | | | | | 92(1999) |
| ORF 1 | Bacillus circulans | L03425 | AAA22259.1 | P48843 | SEQ ID NO: 10 |
| | | | | | of WO |
| | | | | | 00/47711 |
| ORF | Bacillus halodurans | AP001514 | BAB05742.1 | Q9KBA5 | Takami et al, |
| BH2023 | | NC_002570 | NP_242889.1 | | Extremophiles |
| | | | | | 3 (1), 21-28 |
| | | | | | (1999) |
| ORF yvfO | Bacillus subtilis | Z94043 | CABO8009.1 | O07013 | SEQ ID NO: 14 |
| | | Z99121 | CAB15417.1 | O07013 | of WO |
| | | | | O32260 | 00/47711 |
| YvfO | Bifidobacterium | AE014643 | AAN24099.1 | | Schell et al, |
| | longum | NC_004307 | NP_695463.1 | | Proc. Natl. |
| | | | | | Acad. Sci. |
| | | | | | U.S.A. 99 (22), |
| | | | | | 14422-14427 |
| | | | | | (2002) |
| galacta- | Cellvibrio japonicus | X91885 | CAA62990.1 | P48841 | Braithwaite et |
| nase | (Pseudomonas | | | | al, |
| | cellulosa) | | | | Biochemistry |
| | | | | | 36: 15489- |
| | | | | | 15500 (1997) |
| ORF | Clostridium | AE007755 | AAK80519.1 | Q97G04 | Nolling et al, J. |
| CAC2570 | acetobutylicum | | | | Bacteriol. 183 |
| | | | | | (16), 4823- |
| | | | | | 4838 (2001) |
| ORF | Thermotoga maritima | AE001777 | AAD36276.1 | Q9X0S8 | Nelson at al, |
| TM1201 | | NC_000853 | NP_229006.1 | | Nature |
| | | | | | 399: 323- |
| | | | | | 329(1999) |
| Sequence | Myceliophthora | AAE73520 | AAE73520.1 | | U.S. Pat. No. 6,242,237 |
| 2 from | thermophila | | | | |
| patent U.S. | | | | | |
| Pat. No. 6,242,237 | | | | | |
| Sequence | Humicola insolens | AAE73521 | AAE73521.1 | | U.S. Pat. No. 6,242,237 |
| 4 from | | | | | |
| patent U.S. | | | | | |
| Pat. No. 6,242,237 | | | | | |
| ORF GalA | Xanthomonas | AE011762 | AAM36180.1 | | da Silva et al, |
| | axonopodis pv. citri | NC_003919 | NP_641644.1 | | Nature 417 |
| | | | | | (6887), 459- |
| | | | | | 463 (2002) |
| ORF | Xanthomonas | AE011684 | AAM35464.1 | | da Silva et al, |
| XAC0575 | axonopodis pv. citri | NC_003919 | NP_640928.1 | | Nature 417 |
| | | | | | (6887), 459- |
| | | | | | 463 (2002) |
| ORF GalA | Xanthomonas | AE012224 | AAM40555.1 | | da Silva et al, |
| | campestris pv. | NC_003902 | NP_636631.1 | | Nature 417 |
| | campestris | | | | (6887), 459- |
| | | | | | 463 (2002) |
| ORF GalA | Xanthomonas | AE012483 | AAM42894.1 | | da Silva et al, |
| | campestris pv. | NC_003902 | NP_638970.1 | | Nature 417 |
| | campestris | | | | (6887), 459- |
| | | | | | 463 (2002) |
| ORF | Yersinia pestis | AJ414145 | CAC89700.1 | Q8ZHN7 | Parkhill et al, |
| YPO0853 | | NC_003143 | NP_404474.1 | | Nature |
| | | | | | 413: 523- |
| | | | | | 527(2001) |
| ORF | Yersinia pestis | AE013925 | AAM86788.1 | | Deng et al J. |
| Y3238 | | NC_004088 | NP_670537.1 | | Bacteriol. 184 |
| | | | | | (16), 4601- |
| | | | | | 4611 (2002) |

Additional examples are the galactanases derived from Meripilus giganteus (WO 97/32013), Pseudomonas fluorescens, Bacillus agaradhaerens (WO 00/47711), and Bacillus licheniformis (WO 00/47711).

The galactanase may, e.g., be derived from any of the above-mentioned strains. Variants of galactanases of Glycoside hydrolase family 53 are disclosed in patent application DK 2002 01968 filed 20.12.2002. In particular embodiments, the variants are derived from Myceliophthora thermophila, Humicola insolens, Aspergillus aculeatus, or Bacillus licheniformis. Preferred galactanase variants are derived from Myceliophthora thermophila (mature peptide corresponding to amino acids 1-332 of SEQ ID NO: 16). In a specific embodiment, the variants have a degree of identity to amino acids 1-332 of SEQ ID NO: 16 of at least 75%.

Definitions, specific conditions, parameters, as well as particular embodiments of these preferred galactanases forming part of the composition of the invention are apparent from the general polypeptide section hereof (just replace “polypeptide” with “galactanase”). This is for example the case for the calculation of percentage identity, and for the selection of hybridization conditions.

### Polypeptides having Mannanase Activity

The term mannanase as used herein means an enzyme catalyzing the random hydrolysis of 1,4-beta-D-mannosidic linkages in mannans, galactomannans, glucomannans, and galactoglucomannans. The official name is mannan endo-1,4-beta-mannosidase. Alternative name(s) are beta-mannanase, and endo-1,4-mannanase. The EC number according to IUBMB Enzyme Nomenclature is EC 3.2.1.78.

In particular embodiments, the mannanase for use in the composition of the invention i) is classified or can be classified as EC 3.2.1.78; and/or ii) is or can be classified as a Glycoside Hydrolase of family 26, 44, or 5.

The mannanase may, e.g., be derived from strains of Aspergillus (e.g. Aspergillus aculeatus, see WO 94/25576 and U.S. Pat. No. 5,795,764), from strains of Bacillus (WO 91/18974, WO 99/6573, WO 99/64619), strains of Trichoderma (WO 93/24622), strain CBS 480.95 (WO 95/35362), or from the mannanase sequences disclosed at http://afmb.cnrs-mrs.fr/˜cazy/CAZY/index.html as membes of Glycoside Hydrolase family 26, 44 or 5, such as, e.g., SWISS-PROT P55296, MANA_PIRSP; P49424, MANA_PSEFL; P49425, MANA_RHOMR; P51529, MANA_STRLI; P16699, MANB_BACSM; P55278, MANB_BACSU; P22533, MANB_CALSA; P55297, MANB_PIRSP; P55298, MANC_PIRSP.

In particular embodiments, the thermostable mannanase variants are derived from any of the sequences referred to above. Preferred variants are derived from the Aspergillus aculeatus mannanase (WO 94/25576 and U.S. Pat. No. 5,795,764), from strains of Bacillus (WO 91/18974, WO 99/6573, WO 99/64619), from strains of Trichoderma (WO 93/24622), or from strain CBS 480.95 (WO 95/35362). In a specific embodiment, the variants have a degree of identity to the parent mannanase from which it derives of at least 75%.

Definitions, specific conditions, parameters, as well as particular embodiments of these preferred mannanases forming part of the composition of the invention are apparent from the general polypeptide section hereof (just replace “polypeptide” with “mannanase”). This is for example the case for the calculation of percentage identity, and for the selection of hybridization conditions.

### Polypeptides having Dextranase Activity

The term dextranase as used herein means an enzyme catalyzing the endohydrolysis of 1,6-alpha-D-glucosidic linkages in dextran. The official name is dextranase. An alternative Name is alpha-1,6-glucan-6-glucanohydrolase. The number according to the IUBMB Enzyme Nomenclature is 3.2.1.11.

In a particular embodiment the dextranase for use in the composition of the invention is i) is or can be classified as EC 3.2.1.11; and/or ii) is or can be classified as Glycoside Hydrolase family 49, or 66.

The dextranase may, e.g., be derived from Paecilomyces lilacinus (U.S. Pat. No. 6,156,553) or from the dextranase sequences disclosed at http://afmb.cnrs-mrs.fr/˜cazy/CAZY/index.html as membes of Glycoside Hydrolase family 49 or 66, such as, e.g., SWISS-PROT P70744, DEXT_ARTGO; P39652, DEXT_ARTSP; P48845, DEXT_PENMI; P39653, DEXT_STRDO; Q54443, DEXT_STRMU; Q59979, DEXT_STRSL.

In particular embodiments, the thermostable dextranase variants are derived from any of the sequences referred to above. Preferred variants are derived from the Paecilomyces lilacinus (U.S. Pat. No. 6,156,553) dextranase. In a specific embodiment, the variants have a degree of identity to this dextranase of at least 75%.

Definitions, specific conditions, parameters, as well as particular embodiments of these preferred dextranases forming part of the composition of the invention are apparent from the general polypeptide section hereof (just replace “polypeptide” with “dextranase”). This is for example the case for the calculation of percentage identity, and for the selection of hybridization conditions.

Polypeptides having Alpha-galactosidase Activity

Alpha-galactosidases are enzymes that catalyze the following reaction: Melibiose+H(2)O<=>galactose+glucose. The official name is alpha-galactosidase. An alternative name is melibiase. It also hydrolyzes alpha-D-fucosides. The number according to the IUBMB Enzyme Nomenclature is 3.2.1.22.

In particular embodiments, the alpha-galactosidase of the composition of the invention i) is or can be classified as EC 3.2.1.22; and/or ii) is or can be classified as Glycoside Hydrolase family 27, 36, 4, or 57.

The alpha-galactosidase may, e.g., be derived from a strain of Aspergillus (e.g. Aspergillus niger, see e.g. U.S. Pat. No. 6,197,455) or from the alpha-galactosidase sequences disclosed at http://afmb.cnrs-mrs.fr/˜cazy/CAZY/index.html as membes of Glycoside Hydrolase family 27, 36, 4, or 57, such as, e.g., SWISS-PROT P43467, AGA1_PEDPE; P43469, AGA2_PEDPE; P28351, AGAL_ASPNG; O34645, AGAL_BACSU; Q42656, AGAL_COFAR; P14749, AGAL_CYATE; P06720, AGAL_ECOLI; Q9X4Y0, AGAL_RHIME; P30877, AGAL_SALTY; P27756, AGAL_STRMU; Q9UUZ4, AGLC_ASPNG; P04824, MEL1_YEAST; P41945, MEL2_YEAST; P41946, MEL5_YEAST; P41947, MEL6_YEAST; P16551, RAFA_ECOLI.

In particular embodiments, the thermostable alpha-galactosidase variants are derived from any of the sequences referred to above. Preferred variants are derived from the Aspergillus niger (U.S. Pat. No. 6,197,455) alpha-galactosidase. In a specific embodiment, the variants have a degree of identity to this alpha-galactosidase of at least 75%.

Definitions, specific conditions, parameters, as well as particular embodiments of these preferred alpha-galactosidases forming part of the composition of the invention are apparent from the general polypeptide section hereof (just replace “polypeptide” with “alpha-galactosidase”). This is for example the case for the calculation of percentage identity, and for the selection of hybridization conditions.

### Homologous Polypeptides

The present invention refers to polypeptides having an amino acid sequence which has a certain degree of identity to a specified amino acid sequence, and which have enzymatic activity, e.g. endoglucanase, xylanase phytase, protease, galactanase, mannanase, dextranase, or alpha-galactosidase activity (hereinafter “homologous polypeptides”).

For purposes of the present invention the degree of identity between two amino acid sequences, as well as the degree of identity between two nucleotide sequences, is determined by the program “align” which is a Needleman-Wunsch alignment (i.e. a global alignment). The program is used for alignment of polypeptide, as well as nucleotide sequences. The default scoring matrix BLOSUM50 is used for polypeptide alignments, and the default identity matrix is used for nucleotide alignments. The penalty for the first residue of a gap is −12 for polypeptides and −16 for nucleotides. The penalties for further residues of a gap are −2 for polypeptides, and −4 for nucleotides.

“Align” is part of the FASTA package version v20u6 (see W. R. Pearson and D. J. Lipman (1988), “Improved Tools for Biological Sequence Analysis”, PNAS 85:2444-2448, and W. R. Pearson (1990) “Rapid and Sensitive Sequence Comparison with FASTP and FASTA,” Methods in Enzymology 183:63-98). FASTA protein alignments use the Smith-Waterman algorithm with no limitation on gap size (see “Smith-Waterman algorithm”, T. F. Smith and M. S. Waterman (1981) J. Mol. Biol. 147:195-197).

In particular embodiments, the polypeptide has the relevant enzymatic activity, and has an amino acid sequence which has a degree of identity to a specified amino acid sequence (a mature polypeptide) of at least about 65%, or of at least about 70%, or of at least about 75% or of at least about 80%, or of at least about 85%, or of at least about 90%, or of at least about 95%, or of at least about 97%.

In another particular embodiment, these homologous polypeptides have an amino acid sequence which differs by five, four, three, two or only one amino acid(s) from the specified amino acid sequence.

In a particular embodiment, at least one of the enzymes forming part of the composition of the invention has a pH-optimum in the range of 3 to 7 at a temperature of 37° C.

### Allelic Variants and Fragments

The polypeptides referred to herein may comprise the amino acid sequence specified, or they may be an allelic variant thereof; or a fragment thereof that has the relevant enzyme activity. In one embodiment, the polypeptides comprise the amino acid sequence specified or an allelic variant thereof; or a fragment thereof that has the relevant enzyme activity. In another embodiment, the polypeptides consist of the amino acid sequence specified, or an allelic variant thereof; or a fragment thereof that has the relevant enzyme activity.

A fragment of a specified amino acid sequence is a polypeptide having one or more amino acids deleted from the amino and/or carboxyl terminus of this amino acid sequence. In one embodiment, a fragment contains at least 60 amino acid residues, or at least 68, or at least 70, or at least 75, or at least 100, or at least 150, or at least 160, or at least 170, or at least 180, or at least 190, or at least 200, or at least 210, or at least 220, or at least 240, or at least 260, or at least 280, or at least 300, or at least 310, or at least 320, or at least 330, or at least 334, or at least 350, or at least 375, or at least 400, or at least 425, or at least 430 amino acid residues.

An allelic variant denotes any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

A mature polypeptide or a mature amino acid sequence refers to that part of an amino acid sequence which remains after a potential signal peptide part has been cleaved off. And analogously, a mature polypeptide encoding part of a gene refers to that part of a gene, which corresponds to a mature polypeptide.

### Hybridization

The present invention also refers to polypeptides having a specified enzyme activity and which are encoded by nucleic acid sequences which hybridize under very low stringency conditions, preferably low stringency conditions, more preferably medium stringency conditions, more preferably medium-high stringency conditions, even more preferably high stringency conditions, and most preferably very high stringency conditions with a nucleic acid probe which hybridizes under the same conditions with a specified nucleotide sequence, or a subsequence or a complementary strand thereof (J. Sambrook, E. F. Fritsch, and T. Maniatus, 1989, Molecular Cloning, A Laboratory Manual, 2d edition, Cold Spring Harbor, N.Y.). In one particular embodiment the nucleic acid probe is selected from amongst the specified nucleic acid sequences.

A subsequence may be at least 100 nucleotides, or in another embodiment at least 200 nucleotides. Moreover, the subsequence may encode a polypeptide fragment that has the relevant enzyme activity.

For long probes of at least 100 nucleotides in length, very low to very high stringency conditions are defined as prehybridization and hybridization at 42° C. in 5×SSPE, 0.3% SDS, 200 μg/ml sheared and denatured salmon sperm DNA, and either 25% formamide for very low and low stringencies, 35% formamide for medium and medium-high stringencies, or 50% formamide for high and very high stringencies, following standard Southern blotting procedures.

For long probes of at least 100 nucleotides in length, the carrier material is finally washed three times each for 15 minutes using 2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

For short probes which are about 15 nucleotides to about 70 nucleotides in length, stringency conditions are defined as prehybridization, hybridization, and washing post-hybridization at 5° C. to 10° C. below the calculated Tₘ using the calculation according to Bolton and McCarthy (1962, Proceedings of the National Academy of Sciences USA 48:1390) in 0.9 M NaCl, 0.09 M Tris-HCl pH 7.6, 6 mM EDTA, 0.5% NP40, 1×Denhardt's solution, 1 mM sodium pyrophosphate, 1 mM sodium monobasic phosphate, 0.1 mM ATP, and 0.2 mg of yeast RNA per ml following standard Southern blotting procedures.

For short probes which are about 15 nucleotides to about 70 nucleotides in length, the carrier material is washed once in 6×SCC plus 0.1% SDS for 15 minutes and twice each for 15 minutes using 6×SSC at 5° C. to 10° C. below the calculated Tₘ.

### Enzyme Variants

Still further, the polypeptides referred to herein may be variants of the polypeptides specified comprising a substitution, deletion, and/or insertion of one or more amino acids. In a particular embodiment, the polypeptides are thermostable variants of the polypeptides specified.

The amino acid sequences of the variant polypeptides may differ from the amino acid sequence specified by an insertion or deletion of one or more amino acid residues and/or the substitution of one or more amino acid residues by different amino acid residues. Preferably, amino acid changes are of a minor nature, that is conservative amino acid substitutions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter the specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly as well as these in reverse.

### Microbial Sources

The polypeptides referred to herein may be encoded by a nucleotide sequence derived from a naturally occurring microorganism, or they may be an analogue, a fragment, a variant, a mutant, or a synthetic polypeptide, which is amended as compared to the one or more wild-type polypeptide(s) on the basis of which it has been designed (genetically engineered). Synthetic or genetically engineered polypeptides, including shuffled enzymes and consensus enzymes, can be prepared as is generally known in the art, eg by Site-directed Mutagenesis, by PCR (using a PCR fragment containing the desired mutation as one of the primers in the PCR reactions), or by Random Mutagenesis. The preparation of consensus proteins is described in eg EP 897985.

The polypeptides referred to herein may be produced or expressed in the original wild-type microbial strain, e.g. in a strain of Thermoascus aurantiacus, or in another microbial strain, in a plant, or in an animal—as is generally known in the art. E.g., the xylanase and endoglucanase may be co-expressed in one and the same expression host. Also additional enzymes, if any, may be co-expressed.

Accordingly, the polypeptides referred to herein may be wild-type or naturally occurring polypeptides, or they may be genetically engineered or synthetic polypeptides. They may be expressed in an original, wild-type strains or by recombinant gene technology in any other host cell.

Examples of a bacterial polypeptide are a gram positive bacterial polypeptide such as a Bacillus polypeptide, or a Streptomyces polypeptide; or a gram negative bacterial polypeptide, e.g., an E. coli or a Pseudomonas sp. polypeptide.

Examples of a Bacillus polypeptide are a Bacillus agaradhaerens, Bacillus circulans, Bacillus licheniformis, Bacillus pumilus, or Bacillus subtilis polypeptide.

Examples of a Streptomyces polypeptide are a Streptomyces coelicolor, Streptomyces lividans, Streptomyces olivaceoviridis, Streptomyces thermocyaneoviolaceus, Streptomyces thermoviolaceus, or Streptomyces viridosporus polypeptide.

Examples of a fungal polypeptide are a yeast polypeptide such as a Candida, Kluyveromyces, Pichia, Saccharomyces, Schizosaccharomyces, or Yarrowia polypeptide, for example a Pichia stipitis polypeptide; or a filamentous fungal polypeptide such as an Acremonium, Aspergillus, Aureobasidium, Cryptococcus, Emericella, Filibasidium, Fusarium, Gaeumannomyces, Humicola, Lentinula, Magnaporthe, Mucor, Myceliophthora, Neocallimastix, Neurospora, Nocardiopsis, Paecilomyces, Penicillium, Piromyces, Schizophyllum, Talaromyces, Thermoascus, Thermomyces, Thielavia, Tolypocladium, or Trichoderma polypeptide.

In one embodiment, the polypeptide is an Aspergillus aculeatus, Aspergillus awamori, Aspergillus foetidus, Aspergillus japonicus, Aspergillus kawachil, Aspergillus nidulans, Aspergillus niger, Aspergillus oryzae, Aspergillus tubigensis, Emericella nidulans, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium oxysporum f. sp. lycopersici, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Gaeumannomyces graminis, Humicola grisea var. thermoidea, Humicola insolens, Humicola lanuginosa, Lentinula edodes, Magnaporthe grisea, Mucor miehei, Myceliophthora thermophila, Neocallimastix frontalis, Neocallimastix patriciarum, Neurospora crassa, Nocardiopsis dassonvillei, Paecilomyces varioti Bainier, Penicillium funiculosum, purpurogenum, Schizophyllum commune, Talaromyces emersonil, Thermoascus aurantiacus, Thermomyces lanuginosus, Trichoderma harzianum, Trichoderma koningii, Trichoderma longibrachiatum, Trichoderma reesei, Trichoderma terrestris, or Trichoderma viride polypeptide.

It will be understood that the definition of the aforementioned species includes both the perfect and imperfect states, and other taxonomic equivalents e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

Furthermore, such polypeptides may be identified and obtained from other sources including microorganisms isolated from nature (e.g., soil, composts, water, etc.) using the above-mentioned probes. Techniques for isolating microorganisms from natural habitats are well known in the art. The nucleic acid sequence may then be derived by similarly screening a genomic or cDNA library of another microorganism. Once a nucleic acid sequence encoding a polypeptide has been detected with the probe(s), the sequence may be isolated or cloned by utilizing techniques which are known to those of ordinary skill in the art (see, e.g., Sambrook et al, 1989, supra).

### Enzyme Purity

In a particular embodiment of the composition of the invention, at least one of the component polypeptides is isolated, i.e. essentially free of other polypeptides of enzyme activity, e.g., at least about 20% pure, preferably at least about 40% pure, more preferably about 60% pure, even more preferably about 80% pure, most preferably about 90% pure, and even most preferably about 95% pure, as determined by SDS-PAGE. As it is generally known in the art, for detection purposes the SDS-gel can be stained with Coomassie or silver staining. It should be ensured that overloading has not occurred, e.g. by checking linearity by applying various concentrations in different lanes on the gel.

In another embodiment, at least one of the component polypeptides is well-defined. The term well-defined refers to a preparation of the polypeptide in question which is at least 50% pure as determined by Size-exclusion chromatography. In other particular embodiments the preparation is at least 60, 70, 80, 85, 88, 90, 92, 94, or at least 95% pure as determined by this method. As it is generally known in the art, following Size-exclusion chromatography, polypeptides can be detected by measuring absorbance at 214 and/or 280 nm.

In still another embodiment, at least one of the component polypeptides is pure, the term pure indicating, that a fractionation of the polypeptide preparation on an appropriate Size-exclusion column reveals only one major polypeptide component having the enzyme activity in question.

The skilled worker will know how to select an appropriate Size-exclusion chromatography column. He might start by fractionating the preparation on e.g. a HiLoad26/60 Superdex75pg column from Amersham Pharmacia Biotech. If the peaks would not be clearly separated he would try different columns (e.g. with an amended column particle size and/or column length), and/or he would amend the sample volume. By simple and common trial-and-error methods he would thereby arrive at a column with a sufficient resolution (clear separation of peaks), on the basis of which the purity calculation can be performed.

In a particular embodiment at least one polypeptide of the composition of the invention is isolated and/or well-defined and/or pure. In another embodiment at least two of the polypeptides of the composition, are isolated and/or well-defined and/or pure. In a most preferred embodiment each of the thermostable component polypeptides of the composition is isolated and/or well-defined and/or pure.

The use of an isolated and/or well-defined and/or pure polypeptide in the composition of the invention is advantageous. For instance, it is much easier to dose correctly, e.g. to animal feed, enzymes that are essentially free from interfering or contaminating other enzymes. The term dose correctly refers in particular to the objective of obtaining consistent and constant animal feeding results, and the capability of optimising dosage based upon the desired effect.

The composition of the invention can be used for many purposes, for example in animal feed. For such purposes it can be (a) added directly to animal feed (or used directly in a treatment process of vegetable proteins), or (b) it can be used in the production of one or more intermediate compositions such as feed additives or premixes that is subsequently added to feed (or used in a treatment process). The purity indications described above in relation to the terms isolated, well-defined and pure refers to the purity of the component polypeptides, i.e. before these are mixed to form a composition of the invention, and whether this composition is used according to (a) or (b) above.

Polypeptide preparations with purities of this order of magnitude are in particular obtainable using recombinant methods of production, whereas they are not so easily obtained and also subject to a much higher batch-to-batch variation when the polypeptide is produced by traditional fermentation methods.

The polypeptides comprised in the composition of the invention are preferably also purified. The term purified refers to a protein-enriched preparation, in which a substantial amount of low molecular components, typical residual nutrients and minerals originating from the fermentation, have been removed. Such purification can e.g. be by conventional chromatographic methods such as ion-exchange chromatography, hydrophobic interaction chromatography and size exclusion chromatography (see e.g. Protein Purification, Principles, High Resolution Methods, and Applications. Editors: Jan-Christer Janson, Lars Rydén, VCH Publishers, 1989).

### Microorganism Taxonomy

Questions relating to taxonomy are preferably solved by consulting a taxonomy data base, such as the NCBI Taxonomy Browser which is available at the following internet site: http://www.ncbi.nlm.nih.gov/Taxonomy/taxonomyhome.html/. For questions relating to fungal taxonomy, see preferably Dictionary of the Fungi, 9^{th} edition, edited by Kirk, P. M., P. F. Cannon, J. C. David & J. A. Stalpers, CAB Publishing, 2001.

### Compositions and Uses Animal Feed and Animal Feed Additives

Over and above the enzymes described above, the composition of the invention may 6 comprise additional enzymes, vitamins, minerals, and/or additional ingredients, examples of which are listed below.

The composition may be prepared in accordance with methods known in the art, e.g. by mixing of the individual enzyme components, as desired, preferably, in the form of isolated, pure, well-defined, and/or purified enzymes, preferably followed by a formulation step. The formulated composition may be liquid or dry, e.g. in the form of a granulate or a microgranulate. The enzymes may be stabilized in accordance with methods known in the art. At least one compound selected from stabilizers, fillers, pH-regulators, preservatives, viscosity modifying substances, aroma compounds and/or the like ingredients may be added to and mixed with the enzymes. This is so in particular for liquid enzyme compositions.

A preferred use of the composition of the invention is within the field of animal feed.

For the present purposes, the term animal includes all animals, including human beings. In a particular embodiment, the composition of the invention can be used as a feed additive for non-human animals. Examples of animals are non-ruminants, and ruminants, such as cows, sheep and horses. In a particular embodiment, the animal is a non-ruminant animal. Non-ruminant animals include mono-gastric animals, e.g. pigs or swine (including, but not limited to, piglets, growing pigs, and sows); poultry such as turkeys and chicken (including but not limited to broiler chicks, layers); young calves; and fish (including but not limited to salmon).

The terms animal feed, animal feed composition, feed or feed composition mean any compound, preparation, mixture, or composition suitable for, or intended for intake by an animal. The composition of the invention can be fed to the animal before, after, or simultaneously with the diet. The latter is preferred.

The composition of the invention, when intended for addition to animal feed, may be designated an animal feed additive. Such additive may be a relatively simple mixture of the at least two enzymes, preferably in the form of stabilized liquid or dry compositions as referred to above. In another type of animal feed additive the two enzymes are in admixture with other components or ingredients of animal feed. The so-called pre-mixes for animal feed are particular examples of such animal feed additives. Pre-mixes may contain the enzyme(s) in question, and in addition at least one vitamin and/or at least one mineral.

Accordingly, in a particular embodiment, in addition to the component polypeptides, the composition of the invention may comprise at least one fat-soluble vitamin, and/or at least one water-soluble vitamin, and/or at least one trace mineral. The composition may also comprise at least one macro mineral.

Examples of fat-soluble vitamins are vitamin A, vitamin D3, vitamin E, and vitamin K, e.g. vitamin K3.

Examples of water-soluble vitamins are vitamin B12, biotin and choline, vitamin B1, vitamin B2, vitamin B6, niacin, folic acid and panthothenate, e.g. Ca-D-panthothenate.

Examples of trace minerals are manganese, zinc, iron, copper, iodine, selenium, and cobalt.

Examples of macro minerals are calcium, phosphorus and sodium.

Further, optional, feed-additive ingredients are antimicrobial peptides, colouring agents, aroma compounds, and stabilizers.

Examples of antimicrobial peptides (AMP's) are CAP18, Leucocin A, Tritrpticin, Protegrin-1, Thanatin, Defensin, and Ovispirin such as Novispirin (Robert Lehrer, 2000), Plectasins, and Statins, including the compounds and polypeptides disclosed in PCT/KK02/00781 and PCT/DK02/00812, as well as variants or fragments of the above that retain antimicrobial activity.

Examples of antifungal polypeptides (AFP's) are the Aspergillus giganteus, and Aspergillus niger peptides, as well as variants and fragments thereof which retain antifungal activity, as disclosed in WO 94/01459 and WO 02/090384.

In a particular embodiment, the animal feed additive of the invention is intended for being included (or prescribed as having to be included) in animal diets or feed at levels of 0.001-12.0%, or 0.0050-11.0%, or 0.0100-10.0%; more particularly 0.05-5.0%; or 0.2-1.0% (% meaning g additive per 100 g feed). This is so in particular for premixes.

Accordingly, the concentrations of the individual components of the animal feed additive, e.g. the premix, can be found by multiplying the final in-feed concentration of the same component by, respectively, 10-10000; 20-2000; or 100-500 (referring to the above three percentage inclusion intervals).

The final in-feed concentrations of important feed components may reflect the nutritional requirements of the animal, which are generally known by the skilled nutritionist, and presented in publications such as the following: NRC, Nutrient requirements in swine, ninth revised edition 1988, subcommittee on swine nutrition, committee on animal nutrition, board of agriculture, national research council. National Academy Press, Washington, D.C. 1988; and NRC, Nutrient requirements of poultry, ninth revised edition 1994, subcommittee on poultry nutrition, committee on animal nutrition, board of agriculture, national research council. National Academy Press, Washington, D.C. 1994.

The polypeptides forming part of the composition of the invention should of course be applied in animal feed in an effective amount, i.e. in an amount adequate for improving the nutritional value of the feed. It is at present contemplated that each enzyme is administered in the following dosage ranges: 0.01-200; or 0.01-100; or 0.05-100; or 0.05-50; or 0.10-10—all these ranges being in mg enzyme protein per kg feed (ppm).

For determining mg enzyme protein per kg feed, the enzymes are purified from the feed composition, and the specific activity of the purified enzymes is determined using a relevant assay as described above. The enzyme activity of the feed composition as such is also determined using the same assay, and on the basis of these two determinations, the dosage in mg enzyme protein per kg feed is calculated. The same principles apply for determining mg enzyme protein in feed additives.

Of course, if a sample is available of the enzyme used for preparing the feed additive or the feed, the specific activity is determined from this sample (no need to purify the enzymes from the feed composition or the additive).

Animal feed compositions or diets have a relatively high content of protein. An animal feed composition according to the invention has a crude protein content of 50-800, or 75-700, or 100-600, or 110-500, or 120-490 g/kg, and furthermore comprises a composition of the invention.

Furthermore, or in the alternative (to the crude protein content indicated above), the animal feed composition of the invention has a content of metabolisable energy of 10-30, or 11-28, or 11-26, or 12-25 MJ/kg; and/or a content of calcium of 0.1-200, or 0.5-150, or 1-100, or 4-50 g/kg; and/or a content of available phosphorus of 0.1-200, or 0.5-150, or 1-100, or 1-50, or 1-25 g/kg; and/or a content of methionine of 0.1-100, or 0.5-75, or 1-50, or 1-30 g/kg; and/or a content of methionine plus cysteine of 0.1-150, or 0.5-125, or 1-80 g/kg; and/or a content of lysine of 0.5-50, or 0.5-40, or 1-30 g/kg.

Crude protein is calculated as nitrogen (N) multiplied by a factor 6.25, i.e. Crude protein (g/kg)=N(g/kg)×6.25 as stated in Animal Nutrition, 4^{th} edition, Chapter 13 (Eds. P. McDonald, R. A. Edwards and J. F. D. Greenhalgh, Longman Scientific and Technical, 1988, ISBN 0-582-40903-9). The nitrogen content can be determined by the Kjeldahl method (A.O.A.C., 1984, Official Methods of Analysis 14^{th} ed., Association of Official Analytical Chemists, Washington D.C.). But also other methods can be used, such as the so-called Dumas method in which the sample is combusted in oxygen and the amount of nitrous gasses formed are analysed and recalculated as nitrogen.

Metabolisable energy can be calculated on the basis of the NRC publication Nutrient Requirements of Swine (1988) pp. 2-6, and the European Table of Energy Values for Poultry Feed-stuffs, Spelderholt centre for poultry research and extension, 7361 DA Beekbergen, The Netherlands. Grafisch bedrijf Ponsen & Iooijen bv, Wageningen. ISBN 90-71463-12-5.

In a particular embodiment, the animal feed composition of the invention contains at least one vegetable protein or protein source. Examples of vegetable protein or protein sources are soybean, and the cereals such as barley, maize (corn), oat, rice, rye, sorghum and wheat. Preferred cereals are wheat, barley, oats and rye.

In still further particular embodiments, the animal feed composition of the invention contains 0-80% maize; and/or 0-80% sorghum; and/or 0-70% wheat; and/or 0-70% Barley; and/or 0-30% oats; and/or 0-40% soybean meal; and/or 0-10% fish meal; and/or 0-20% whey.

Animal diets can e.g. be manufactured as mash feed (non-pelleted) or pelleted feed. Typically, the milled feed-stuffs are mixed and sufficient amounts of essential vitamins and minerals are added according to the specifications for the species in question, see Example 7 herein.

The composition of the invention can be added in the form of a solid or liquid enzyme formulation, or in the form of a feed additive, such as a pre-mix. A solid composition is typically added before or during the mixing step; and a liquid composition is typically added after the pelleting step. In the process of Example 7, however, the thermostable liquid enzyme composition is added before the pelleting step.

The composition of the invention when added to animal feed leads to an improved nutritional value of the feed, e.g. the growth rate and/or the weight gain and/or the feed conversion (i.e. the weight of ingested feed relative to weight gain) of the animal is/are improved. These results may be due to, in turn, one or more of the following effects: Reduction of the viscosity of materials present in the animal's gut; release of nutrients entrapped e.g. in cell walls of cereals; supplementation and improvement of the endogenous enzyme activities of the animal and the gut microbial flora (this is so in particular in young animals).

In vitro experiments simulating stomach and small intestine in monogastrics have shown that the endoglucanase derived from Thermoascus aurantiacus as described in the experimental part is capable of decreasing the viscosity of the luminal content (barley mill fraction enriched in beta-glucans), thereby enhancing nutrient absorption.

In particular embodiments the weight gain is at least 101, 102, 103, 104, 105, 106, 107, 108, 109, or at least 110% of the control (no enzyme addition).

In further particular embodiments the feed conversion is at most (or not more than) 99, 98, 97, 96, 95, 94, 93, 92, 91 or at most 90%, as compared to the control (no enzyme addition).

The composition of the invention may also be used in vitro, e.g. to treat vegetable proteins. The term vegetable proteins as used herein refers to any compound, composition, preparation or mixture that includes at least one protein derived from or originating from a vegetable, including modified proteins and protein-derivatives. In particular embodiments, the protein content of the vegetable proteins is at least 10, 20, 30, 40, 50, or 60% (w/w).

Examples of vegetable proteins or protein sources are cereals such as barley, wheat, rye, oat, maize (corn), rice, and sorghum. Other examples are soya bean meal, peas and rape seed meal from leguminosae and brassica families.

The vegetable protein or protein source is typically suspended in a solvent, eg an aqueous solvent such as water, and the pH and temperature values are adjusted paying due regard to the characteristics of the enzymes in question. The enzymatic reaction is continued until the desired result is achieved, following which it may or may not be stopped by inactivating the enzymes, e.g. by a heat-treatment step.

In another particular embodiment of a treatment process of the invention, the enzyme actions are sustained, meaning e.g. that the enzymes are added to the vegetable proteins or protein sources, but their activity is so to speak not switched on until later when desired, once suitable reaction conditions are established, or once any enzyme inhibitors are inactivated, or whatever other means may have been applied to postpone the action of the enzymes.

These are additional particular embodiments of the present invention:

A composition comprising i) at least one polypeptide having xylanase activity, the polypeptide being a family 11 glycoside hydrolase; and ii) at least one polypeptide having endoglucanase activity, the polypeptide comprising (a) an amino acid sequence of at least 75% identity to amino acids 1 to 335, or 31 to 335 of SEQ ID NO:2, and/or wherein the polypeptide is (b) encoded by a nucleic acid sequence which hybridizes under low stringency conditions with (i) the mature endoglucanase encoding part of the plasmid contained in Escherichia coli DSM 14541, (ii) nucleotides 1 to 1008, or 90 to 1008 of SEQ ID NO:1, (iii) a subsequence of (i) or (ii) of at least 100 nucleotides, or (iv) a complementary strand of (i), (ii) or (iii); (c) a variant of the polypeptide having the amino acid sequence of SEQ ID NO:2 comprising a substitution, deletion, and/or insertion of one or more amino acids, (d) an allelic variant of (a) or (b), or (e) a fragment of (a), (b), or (d) that has endoglucanase activity;

Any of the above compositions, wherein i) the polypeptide having endoglucanase activity is a family 5 glycoside hydrolase; ii) at least one of the polypeptides having endoglucanase or xylanase activity is thermostable; iii) the polypeptide having xylanase activity is derived from a strain of Aspergillus, Humicola, Thermomyces, or Trichoderma; iv) wherein the composition further comprises at least one polypeptide having endo-1,3(4)-beta-glucanase activity, and/or at least one polypeptide having protease activity, and/or at least one polypeptide having phytase activity; v) wherein at least one of the further polypeptides is thermostable; vi) wherein the composition further comprises (a) at least one fat soluble vitamin, and/or (b) at least one water soluble vitamin, and/or (c) at least one trace mineral, and/or (d) at least one macro mineral; vii) wherein the composition is an animal feed additive;

Any of the above compositions, further comprising at least one polypeptide having endo-1,3(4)-beta-glucanase activity, and/or at least one polypeptide having protease activity, and/or at least one polypeptide having phytase activity, the endoglucanase and/or the xylanase and/or the endo-1,3(4)-beta-glucanase, and/or the phytase, and/or the protease being preferably thermostable, or the xylanase, as well as the endoglucanase and/or the endo-1,3(4)-beta-glucanase being thermostable, or the xylanase, the phytase, and the endoglucanase and/or the endo-1,3(4)-betaglucanase being thermostable.

Compositions comprising (i) at least one polypeptide having xylanase activity, and (ii) at least one polypeptide having endoglucanase activity, wherein at least one of the polypeptides are thermostable; as well as methods of preparing such compositions, their use in animal feed, their use for treatment of vegetable proteins, and animal feed compositions with content thereof. In a particular embodiment both polypeptides are thermostable. In a further preferred embodiment, at least one of an additional polypeptide of the composition, if any, is also thermostable (eg. an endo-1,3(4)-beta-glucanase, a protease, or a phytase).

A method of preparing any of the above compositions, the method comprising the step of mixing the polypeptides having endoglucanse and xylanase activity.

Use of the any of the above compositions in animal feed, in the preparation of animal feed.

A method for improving the nutritional value of an animal feed, wherein any of the above compositions is added to the feed.

An animal feed composition having a crude protein content of 50 to 800 g/kg and comprising any of the above compositions, the feed composition preferably comprising at least one of wheat, barley, oats or rye.

A method for the treatment of vegetable proteins, comprising the step of adding any of the above compositions to at least one vegetable protein or protein source, the vegetable protein source preferably including wheat, barley, oats and/or rye.

### EXAMPLES

### Example 1

### Enzyme Activity Assays

Endoglucanase

This assay is primarily for assaying endoglucanase activity in animal feed in the form of mash feed or pellets, or in enzyme premix in powder form. For assaying the endoglucanase activity of enzyme samples which are neither mixed with feed components, nor with vitamins and minerals and the like as in premix, an appropriate starting point is after the heading “incubation and precipitation.”

Reagents and Solutions

Azo-CM-Cellulose Solution

Suspend 0.4 g Azo-CM-cellulose (Megazyme) in 16 ml of demineralised water and stir thoroughly in a boiling water bath for 5 minutes until complete dissolution. After cooling to room temperature 1 ml of a 2M sodium acetate buffer, pH4.5 (Megazyme) is added. Adjust the volume with water to 20 ml. This solution is kept at 5° C.

Extraction Buffer

Dissolve 5.44 g sodium acetate-trihydrate and 6.24 g sodium dihydrogen-o-phosphate in approximately 900 ml distilled water and adjust pH to 4.2 with 1N HCl. Add distilled water ad 1000 ml.

Precipitation Solution Dissolve 40 g sodium acetate tri-hydrate and 4 g zinc acetate in 150 ml demineralised water, and adjust to pH5.0 with 5M HCl. Add demineralised water ad 200 ml. Add this solution to 800 ml ethanol (95%v/v), mix and store at room temperature in a sealed bottle.

Assay Procedure

Pre-Treatment of Premix

Add 10 g premix to 90 g corn flour and mix well. Add 10 g of this mixture to 90 g corn flour and mix well.

Sample Preparation and Dilution

Weigh 50.0 g feed (or premix pre-treated as described above) into a 500 ml Erlenmeyer flask and add 500 ml Extraction Buffer. Stir for 45 minutes. Take out a sample of 50 ml for centrifugation for 10 minutes at 2000×g. The supernatants are used for the below enzyme reaction, diluted as required with extraction buffer.

Incubation and Precipitation

The incubation temperature is 50° C. 0.1 ml of the substrate is pipetted into each vial and pre-incubated for 5 minutes before adding 0.1 ml of the supernatant from above. After 60 minutes 0.6 ml of the precipitation solution is added to each vial, and the vial is mixed thoroughly on a Vortex mixer. The samples are allowed to stand for 15 minutes at room temperature, and are then mixed again and subjected to centrifugation at 3500 rpm for 10 minutes.

OD Measurements and Activity Calculation

300 microliter of the supernatants from above is immediately pipetted into microtiter plates and the absorbancy at 600 nm is measured. The concentration of endoglucanase in the samples is calculated by reference to an appropriate standard curve.

Xylanase

This assay is primarily for assaying xylanase activity in animal feed in the form of mash feed or pellets, or in enzyme premix in powder form. For assaying the activity of enzyme samples which are neither mixed with feed components, nor with vitamins and minerals and the like as in premix, an appropriate starting point is after the heading “incubation and precipitation.”

Reagents and solutions

Azo-Xylan (Birchwood)

Suspend 0.4 g Azo-Xylan (Birchwood, Megazyme) in 16 ml of demineralised water and stir thoroughly in a boiling water bath for 5 minutes until complete dissolution. Cool to room temperature and add 1 ml 2M sodium acetate buffer, pH 4.5 (Megazyme). Add demineralised water ad 20 ml. Store at 5° C.

Extraction Buffer

Dissolve 5.44 g sodium acetate-trihydrate and 6.24 g sodium dihydrogen-o-phosphate in approximately 900 ml distilled water and adjust pH to 4.2 with 1N HCl. Add distilled water ad 1000 ml.

Precipitation Solution

95% (v/v) laboratory grade ethanol is used as the Precipitation Solution.

Assay Procedure

Pre-Treatment of Premix

Add 10 g premix to 90 g corn flour and mix well. Add 10 g of this mixture to 90 g corn flour and mix well.

Sample Preparation and Dilution

Weigh 50.0 g feed (or premix pre-treated as described above) into a 500 ml Erlenmeyer flask and add 500 ml Extraction Buffer. Stir for 45 minutes. Take out a sample of 50 ml for centrifugation for 10 minutes at 2000×g. The supernatants are used for the below enzyme reaction, diluted as required with extraction buffer.

Incubation and Precipitation

The incubation temperature is 50° C. 0.125 ml of the substrate is pipetted into each vial and pre-incubated for 5 minutes before adding 0.1 ml of the supernatant from above. After 150 minutes 0.64 ml of the precipitation solution is added to each vial, and the vial is mixed thoroughly on a Vortex mixer. The samples are allowed to stand for 15 minutes at room temperature, and are then mixed again and subjected to centrifugation at 3500 rpm for 10 minutes.

OD Measurements and Activity Calculation

300 microliter of the supernatants from above is immediately pipetted into microtiter plates and the absorbancy at 600 nm is measured. The concentration of xylanase in the samples is calculated by reference to an appropriate standard curve.

Endo-1,3(4)-beta-glucanase

This assay is primarily for assaying endo-1,3(4)-beta-glucanase activity in animal feed in the form of mash feed or pellets, or in enzyme premix in powder form. For assaying the activity of enzyme samples which are neither mixed with feed components, nor with vitamins and minerals and the like as in premix, an appropriate starting point is after the heading “incubation and precipitation.”

Reagents and Solutions

Azo-Barley Beta-glucan solution

1% Azo-Barley beta-glucan (Megazyme) is used as a substrate.

Extraction Buffer

Dissolve 5.44 g sodium acetate-trihydrate and 6.24 g sodium dihydrogen-o-phosphate in approximately 900 ml distilled water and adjust pH to 4.2 with 1N HCl. Add distilled water ad 1000 ml.

Precipitation Solution

Dissolve 40 g sodium acetate tri-hydrate and 4 g zinc acetate in 150 ml distilled water, and adjust to pH5.0 with concentrated HCl. Add distilled water ad 200 ml. Add this solution to 800 ml methyl cellosolve (2-methoxyethanol), mix and store at room temperature.

Assay Procedure

Pre-Treatment of Premix

Add 10 g premix to 90 g corn flour and mix well. Add 10 g of this mixture to 90 g corn flour and mix well.

Sample Preparation and Dilution

Weigh 50.0 g feed (or premix pre-treated as described above) into a 500 ml Erlenmeyer flask and add 500 ml Extraction Buffer. Stir for 45 minutes. Take out a sample of 50 ml for centrifugation for 10 minutes at 2000×g. The supernatants are used for the below enzyme reaction, diluted as required with extraction buffer.

Incubation and Precipitation

The incubation temperature is 50° C. 0.1 ml of the substrate is pipetted into each vial and pre-incubated for 5 minutes before adding 0.1 ml of the supernatant from above. After 90 minutes 0.5 ml of the precipitation solution is added to each vial, and the vial is mixed thoroughly on a Vortex mixer. The samples are allowed to stand for 15 minutes at room temperature, and are then mixed again and subjected to centrifugation at 3500 rpm for 10 minutes.

OD Measurements and Activity Calculation

300 microliter of the supernatants from above is immediately pipetted into microtiter plates and the absorbancy at 600 nm is measured. The concentration of endo-1,3(4)-beta-glucanase in the samples is calculated by reference to an appropriate standard curve.

Specific Enzyme Activity

For determining specific enzyme activity, the concentration of enzyme protein can be calculated as follows: a) By measuring the absorbance at 280 nm combined with the theoretical molecular weight and the theoretical molar extinction coefficient (both determined from the amino acid sequence); or b) From amino acid analysis. Both methods require a highly purified enzyme sample with full activity

### Examples 2-5

Reagents, Media, and Equipment

Reagents:

Unless otherwise specified, the chemicals used were commercial products of at least reagent grade.
AZCL-substrates from Megazyme:
Azurine-Cross-Linked substrates are supplied as fine powders which are insoluble in buffered solution, but rapidly hydrate to form gel particles which are readily and rapidly hydrolysed by the relevant enzymes, thus releasing the soluble dye-labeled fragment.
AZCL-Barley-beta-Glucan from Megazyme
AZCL-Oat-Spelt-xylan, AZCL-HE-cellulose, AZCL-Potato-Galactan, AZCL-Galactomannan (carob), AZCL-Tamarind-Xyloglucan, AZCL-Debranched-Arabinan
IPTG (Promega, Cat. No. V3951)
X-gal (Promega, Cat. No. V3941)
LMP agarose (Promega, Cat. No. V2111)
   Media:
Buffer system (pH 3 to pH 11): 100 mM succinic acid, 100 mM HEPES, 100 mM CHES, 100 mM CABS, 1 mM CaCl₂, 150 mM KCl, 0.01% Triton® X-100 adjusted to pH-values 2.0, 2.5, 3.0, 3.5, 4.0, 4.5, 5.0, 6.0, 7.0, 8.0, 9.0, 10.0 or 11.0 with HCl or NaOH (herein for short designated “the succinic acid buffer system”).
WB (Wheat Bran medium):
30 g wheat bran, 45 ml of the following solution in each 500 ml shake flask:
   4 g Yeast Extract
   1 g KH₂PO₄
   0.5 g MgSO₄^{.}7H₂O
   15 g Glucose
   1000 ml Tap water

Autoclave at 121° C. for 20 min.; pH 5.4 after autoclaving

CBH1 medium:

| | | | | | |
|---|---|---|---|---|---|
| Avicel | 25 | g | (NH₄)₂SO₄ | 1.4 | g |
| KH₂PO₄ | 2 | g | Urea | 0.3 | g |
| CaCl₂.2H₂O | 0.3 | g | MgSO₄.7H₂O | 0.3 | g |
| FeSO₄.7H₂O | 5 | mg | MnSO₄.H₂O | 1.6 | mg |
| Peptone | 1 | g | Yeast Extract | 10 | g |
| TWEEN80 | 1 | ml | Glucose | 5 | g |
| H₂O | 1000 | ml | | | |

80 ml in 500 ml Erlenmeyer flask, autoclave 20 minutes under 121° C.
LB liquid medium: To 950 ml of deionized H₂O, add: 10 g bacto-tryptone, 5 g bacto-yeast extract, 10 g NaCl. Shake until the solutes have dissolved. Adjust the pH to 7.0 with 5N NaOH (˜0.2 ml). Adjust the volume of the solution to 1 liter with deionized H₂O. Sterilize by autoclaving for 20 minutes at 15 lb/sq. in. on liquid cycle.
LB plates with ampicillin/IPTG/X-Gal: Add 15 g agar to 1 liter of LB medium. Add ampicillin to a final concentration of 100 μg/ml, then supplement with 0.5mM IPTG and 80 μg/ml X-gal and pour the plates.
1% LMP agarose gel: Add 1 g LMP agarose into 100 ml 1×TAE buffer.
IPTG stock solution (0.1M):
Add distilled water to 1.2 g IPTG to 50 ml final volume, filter-sterilize and store at 4° C.
   Equipment, Including Various Kits:
Resource Q column (Amersham Pharmacia, Anion Exchange)
Superdex75 column (Amersham Pharmacia 17-1047-01)
IEF-gel (Amersham Pharmacia 80-1124-80)
Thermomixer comfort (Eppendorf)
RNeasy Mini Kit (QIAGEN, Cat. No. 74904)
3′ RACE Kit (GIBCO, Cat. No. 18373-019) including Adapter primer, and AUAP
dNTP mix (100 mM, Promega, Cat. No. U1330)
TaqDNA polymerase system (Promega, Cat. No. M1661) including PCR buffer (200 mM Tris-HCl (pH8.4), 500 mM KCl)
PCR Preps DNA Purification System (Promega, Cat. No. A7170)
pGEM-T Vector System (Promega, Cat. No. A3600) including T4 DNA Ligase 2XBuffer
JM109 high efficiency competent cells (Promega, Cat. No. L1001)
Minipreps DNA Purification System (Promega, Cat. No. A7100)
BigDye Terminator Cycle Sequencing Ready Reaction Kit (PE Applied Biosystems, Cat. No. 4303149)
ABI Prism 377 DNA sequencer (PE)
5′RACE system (GIBCO, CAT. NO. 18374-058) including Abridged Anchor Primer

### Example 2

### Cultivation of Thermoascus aurantiacus CGMCC No. 0670

Thermoascus aurantiacus CGMCC No. 0670 was grown in WB medium (30 g/500 ml flask) at 45° C. for 4 days. Enzyme extraction was carried out by adding about 150 ml sterilized water into each shake flask and maintaining at 4° C. for at least 4 hours. Supernatant was collected by centrifugation at 7000 rpm for 20 minutes.

### Example 3

### Purification of Endoglucanase of Thermoascus aurantiacus CGMCC No. 0670

1500 ml supernatant from Example 2 was precipitated with ammonium sulfate (80% saturation) and re-dissolved in 100 ml buffer, ultra-filtrated, and then filtered through a 0.45 m filter. The final volume was 30 ml. The solution was applied to a 6 ml Resource Q column equilibrated in 25 mM Tris-HCl buffer, pH 7.4, and the proteins were eluted with a linear NaCl gradient (0-0.5M). Fractions from the column were analyzed for endoglucanase activity using the below assay at pH 7.0, and 45° C. Fractions with endoglucanase activity were pooled. Then the pooled solution was ultra-filtrated, and the concentrated solution was applied to a Superdex75 column equilibrated with 25 mM Tris-HCl, pH7.4. The proteins were eluted with the same buffer. Endoglucanase-containing fractions were analyzed by SDS-PAGE and pure fractions were pooled.

Endoglucanase Assay

Substrate: AZCL-beta-Glucan (barley)
Temperature: As desired, e.g. 40, 45, or 50° C.
pH: As desired, e.g. pH 3, or pH 7
Assay buffers (unless otherwise indicated):
   200 mM Succinic acid buffer (pH 3)
   200 mM Tris-HCl buffer (pH 7)
      0.4% AZCL-beta-glucan was suspended in buffer with addition of 0.01% Triton X-100 by gentle stirring. Then a limited amount of this suspension and enzyme samples were mixed in a Microtiter plate or Eppendorf tube and placed on ice before reaction (for amount of substrate and enzyme see the below Results section). The assay was initiated by transferring the Microtiter plate/Eppendorf tube to an Eppendorf thermomixer, which was set to the assay temperature. The plate/tube was incubated for 15-30 minutes on the Eppendorf thermomixer at its shaking rate 700 rpm for Microtiter plate and 1400 rpm for Eppendorf tube reaction. The incubation was stopped by transferring the plate/tube back to the ice bath. Then the tube was centrifuged in an icecold centrifuge for a few minutes and 100/200 ml supernatant was transferred to a microtiter plate. OD₅₉₅ was read as a measure of endoglucanase activity. All reactions were done in triplicate and a buffer blind was included in the assay (instead of enzyme).

### Example 4

### Characterization of the Endoglucanase Cel5A of Thermoascus aurantiacus CGMCC No. 0670

Three endoglucanases with different profile (pH, temperature, molecular weight, substrate specificity) were purified from culture broth of Thermoascus aurantiacus CGMCC No. 0670 grown in WB media.

The one showing endoglucanase activity over relatively wide ranges of pH and temperature was selected for further study.

The purified enzyme was blotted onto a PVDF membrane and N-terminal sequenced. The following sequence was obtained:

N-?LVFTSFGSNESGAEFGSQN.

A homology search showed that this is a family 5 glycoside hydrolase. It is therefore designated endoglucanase Cel5A of Thermoascus aurantiacus.

Molecular Weight and pl Determination of Endoglucanase Cel5A

The purity of the purified endoglucanase was verified by SDS-PAGE and IEF gel. The molecular weight of the enzyme is around 32 KDa. Overlay of beta-glucan plate with IEF gel showed that there is only one beta-glucanase activity with pl around 3.5 in the sample.

pH-Profile of Endoglucanase Cel5A at 45° C.

20 ml enzyme sample and 200 ml 0.2% AZCL-beta-glucan in the succinic acid buffer system pH values from pH2.0 to pH11.0 (see above, Media) were mixed in a Microtiter plate and placed on ice before reaction. The assay was initiated by transferring the Microtiter plate to an Eppendorf thermomixer, which was set to the assay temperature 45° C. The plate was incubated for 20 minutes on the Eppendorf thermomixer at 700 rpm shaking rate. The incubation was stopped by transferring the tube back to the ice bath. Then the plate was centrifuged in an ice-cold centrifuge for a few minutes and 100 ml supernatant was transferred to a microtiter plate. OD₅₉₅ was read as a measure of beta-glucanase activity. All reactions were done in triplicate and a buffer blind was included in the assay (instead of enzyme). The results are shown in Table 1 below. In the pH-range of pH 2 to 7, the enzyme retains at least 50% of its maximum activity. The optimum pH is around pH 2.

TABLE 1
pH activity profile

| pH | Activity | Relative Activity |
|---|---|---|
| 2 | 1.198 | 1.000 |
| 3 | 1.150 | 0.960 |
| 4 | 0.987 | 0.824 |
| 5 | 0.839 | 0.700 |
| 6 | 0.810 | 0.676 |
| 7 | 0.631 | 0.527 |
| 8 | 0.218 | 0.182 |
| 9 | 0.135 | 0.112 |
| 10 | 0.101 | 0.084 |
| 11 | 0.063 | 0.053 |

pH 3 Stability of Endoglucanase Cel5A at 40° C.:

150 ml enzyme sample and 300 ml 0.2M Succinic acid buffer pH 3 were mixed in an Eppendorf tube and incubated under 40° C. for 2 hours. Then 100 ml sample was transferred into a new Eppendorf tube with 900 ml 0.4% AZCL-beta-glucan in 0.2M Tris-HCl buffer pH7 with 0.1% Triton X100 and placed on ice before reaction. The assay was initiated by transferring the Eppendorf tube to an Eppendorf thermomixer, which was set to the assay temperature 40° C. The tube was incubated for 30 minutes on the Eppendorf thermomixer at its highest shaking rate (1400 rpm). The incubation was stopped by transferring the tube back to the ice bath. Then the tube was centrifuged in an icecold centrifuge for a few minutes and 200 ml supernatant was transferred to a microtiter plate. OD₅₉₅ was read as a measure of endoglucanase activity. All reactions were done in triplicate and a buffer blind was included in the assay (instead of enzyme). For blank, the same amount of substrate, buffer and enzyme were mixed just before reaction start. The results are shown in Table 2 below. It appears that there is no substantial loss of activity after incubation at pH 3 for 2 hours at 40° C.

TABLE 2
pH 3 stability

| Treatment | Activity | Relative activity |
|---|---|---|
| pH 3 incubation | 0.332 | 1.081 |
| no incubation | 0.307 | 1.000 |

Temperature Profile of Endoglucanase Cel5A at pH 7:

200 ml 0.4% AZCL-beta-glucan in 0.2M Tris-HCl buffer pH 7 and 30 ml enzyme sample were mixed in an Eppendorf tube and put on ice before reaction. The assay was initiated by transferring the Eppendorf tube to an Eppendorf thermomixer, which was set to the assay temperature from 15, 20, 30, 40, 50, 60, 70, 80° C. The tube was incubated for 30 minutes on the Eppendorf thermomixer at its highest shaking rate (1400 rpm). The incubation was stopped by transferring the tube back to the ice bath. Then the tube was centrifuged in an icecold centrifuge for a few minutes and 200 ml supernatant was transferred to a microtiter plate. OD₅₉₅ was read as a measure of endoglucanase activity. All reactions were done in triplicate and a buffer blind was included in the assay (instead of enzyme). The results are shown in Table 3 below, from which it appears that the enzyme is active within the whole temperature range of 20 to 80° C. The optimum temperature is around 70° C. At 40° C. and 80° C., the relative activity is 58% and 37%, respectively (relative to the activity at 70° C.).

TABLE 3
Temperature activity profile

| Temperature (° C.) | Activity | Relative Activity |
|---|---|---|
| 14 | 0.463 | 0.188 |
| 20 | 0.499 | 0.202 |
| 30 | 0.832 | 0.338 |
| 40 | 1.428 | 0.580 |
| 50 | 1.992 | 0.808 |
| 60 | 2.202 | 0.894 |
| 70 | 2.464 | 1.000 |
| 80 | 0.915 | 0.371 |

Thermostability at 50, 60, 70 and 85° C. of Endoglucanase Cel5A at pH 7.4:

100 ml enzyme sample (pH7.4) in an Eppendorf tube was incubated for 10 and 20 minutes on the Eppendorf Thermomixer at 50, 60, 70° C. and 300 rpm shaking. For stability at 85° C., the same method was applied but with sampling time as 0, 2, 5 and 10 minutes. The incubation was stopped by transferring the tube back to the ice bath. Un-incubated sample was used as control. The 30 ml of the above incubated sample was transferred into a new Microtiter plate and 200 ml 0.4% AZCL-beta-glucan in 0.2M Tris-HCl buffer pH 7 was added. The assay was initiated by transferring the Microtiter plate to an Eppendorf thermomixer, which was set to the assay temperature 40° C. The plate was incubated for 30 minutes on the Eppendorf thermomixer at 700 rpm shaking rate. The incubation was stopped by transferring the tube back to the ice bath. Then the plate was centrifuged in an icecold centrifuge for a few minutes and 100 ml supernatant was transferred to a microtiter plate. OD₅₉₅ was a measure of endoglucanase activity. All reactions were done in triplicate and a buffer blind was included in the assay (instead of enzyme). The results are shown in Table 4 (50-70° C.), and in Table 5 (850° C.). The enzyme appears to fully retain its activity after having been incubated for 10 to 20 minutes at a temperature in the range of 50 to 70° C. Also after incubation at 85° C. for 10 minutes, the enzyme seems to fully retain its activity.

TABLE 4
Thermostability at 50, 60 and 70° C.

| Temperature/ | Activity | | | Relative Activity | | |
|---|---|---|---|---|---|---|
| Time (minutes) | 50° C. | 60° C. | 70° C. | 50° C. | 60° C. | 70° C. |
| 0 | 0.884 | 0.884 | 0.884 | 1.000 | 1.000 | 1.000 |
| 10 | 0.790 | 0.779 | 0.784 | 0.894 | 0.882 | 0.888 |
| 20 | 0.730 | 0.867 | 0.920 | 0.826 | 0.981 | 1.041 |

TABLE 5
Thermostability at 85° C.

| Time (minutes) | Activity | Relative Activity |
|---|---|---|
| 0 | 0.444 | 1.000 |
| 2 | 0.552 | 1.242 |
| 5 | 0.523 | 1.178 |
| 10 | 0.457 | 1.029 |

Substrate Specificity of Endoglucanase Cel5A at pH 3 and 50° C. on Various Cellulase and Hemicellulase Substrates:

400 ml 0.2% AZCL-substrate (xylan, HE-cellulose, Galactan, Mannan, Xyloglucan, Arabinan) in 0.2M succinic acid buffer pH 3 with 0.01% Triton X100 and 30 ml enzyme sample (5×dilution by 0.2 M succinic acid buffer) were mixed in an Eppendorf tube and put on ice before reaction. The assay was initiated by transferring the Eppendorf tube to an Eppendorf thermomixer, which was set to the assay temperature 50° C. The tube was incubated for 15 minutes on the Eppendorf thermomixer at its highest shaking rate (1400 rpm). The incubation was stopped by transferring the tube back to the ice bath. Then the tube was centrifuged in an icecold centrifuge for a few minutes and 200 ml supernatant was transferred to a microtiter plate. OD₅₉₅ was read as a measure of endoglucanase activity. All reactions were done in triplicate and a buffer blind was included in the assay (instead of enzyme). From the results which are shown in Table 6, it appears that the enzyme can degrade beta-glucan and HE-Cellulose, but it has no or very low activity on xylan, arabinan, mannan, xyloglucan.

TABLE 6
Substrate specificity

| Enzyme | | | | | | |
|---|---|---|---|---|---|---|
| Activity/ | | | | | | |
| Sample | Beta | Xylo- | | | | |
| no. | glucanase | glucanase | Mannanase | Arabinase | Xylanase | Cellulase |
| 1 | 2.438 | 0.051 | 0.082 | 0.155 | 0.112 | 2.136 |
| 2 | 3.102 | 0.056 | 0.087 | 0.149 | 0.116 | 2.503 |
| 3 | 3.144 | 0.054 | 0.082 | 0.155 | 0.115 | 2.948 |
| Mean | 2.895 | 0.054 | 0.084 | 0.153 | 0.114 | 2.529 |

### Example 5

### Cloning of the Gene Encoding Endoglucanase Cel5A of Thermoascus aurantiacus CGMCC 0670

The gene fragment encoding endoglucanase Cel5A was cloned by RT-PCR from Thermoascus aurantiacus CGMCC 0670 as described below.

Sequence analysis of the cDNA clone showed that the sequence contains a coding region of 1005 nucleotides (SEQ ID NO: 1). The translation product having SEQ ID NO: 2 is 335 amino acids in length. Expectedly, amino acid residues 1 to 30 constitute a signal-peptide part, and amino acid residues 31 to 335 constitutes the catalytic domain.

Cultivation and Isolation of Mycelium

Thermoascus aurantiacus CGMCC 0670 was grown in CBH1 medium at 45° C. and 165 rpm for 3 days. Then the mycelium was harvested by centrifugation at 7000 rpm for 30 minutes. Harvested mycelium was stored at minus 80° C. before being used for extraction of 10 RNA.

Extraction of Total RNA

The total RNA was extracted from 100 mg of the mycelium isolated above using the RNeasy Mini Kit.

Design of Degenerate Primers

Degenerate primers were designed based on determined N-terminal amino acid sequence N-?LVFTSFGSNESGAEFGSQN (SEQ ID NO: 3).

(SEQ ID NO: 4)

1: 5′ AA(T/C) GA(A/G) TC(T/C/A/G) GG(T/C/A/G)
GC(T/C/A/G) GAA TT 3′

(SEQ ID NO: 5)

2: 5′ AA(T/C) GA(A/G) TC(T/C/A/G) GG(T/C/A/G)
GC(T/C/A/G) GAG TT 3′

(SEQ ID NO: 6)

3: 5′ AA(T/C) GA(A/G) AG(T/C) GG(T/C/A/G)
GC(T/C/A/G) GAA TT 3′

(SEQ ID NO: 7)

4: 5′ AA(T/C) GA(A/G) AG(T/C) GG(T/C/A/G)
GC(T/C/A/G) GAG TT 3′

Cloning of the 3′ End of the Endoglucanase

The 3′ RACE kit was used to synthesize the cDNA of the endoglucanase. About 5 mg total RNA was used as template and the Adapter Primer (provided by the 3′RACE system) was used to synthesize the first strand of cDNA. Then the cDNA was amplified by using different degenerate primers. The PCR reaction system and conditions were as follows:

| | | |
|---|---|---|
| 10x PCR buffer | 5 | μl |
| 25 mM MgCl₂ | 3 | μl |
| 10 mM dNTP mix | 1 | μl |
| 3′Primer (10 μM) | 1 | μl |
| AUAP (10 μM, provided by 3′RACE system) | 1 | μl |
| TaqDNA polymerase (5 u/μl, Promega) | 0.5 | μl |
| cDNA synthesis reaction | 2 | μl |
| Add autoclaved, distilled water to | 50 | μl |

Conditions:

| | | |
|---|---|---|
| 94° C. | 3 min | |
| 94° C. | 40 sec | |
| 55° C. | 40 sec | 30 cycles |
| 72° C. | 1 min | |
| 72° C. | 10 min | |

Gel analysis of the PCR product gave a specific band about ˜1 kb fragment using primer 2 and primer 3, and the products were recovered from 1% LMP agarose gel, and purified by incubation at 70° C. followed by using PCR Preps DNA Purification System. The concentration of the purified products was determined by measuring the absorbances A₂₆₀ and A₂₈₀ in a spectrophotometer. Then these purified fragments were ligated to pGEM-T Vector (Promega kit, Cat. No. A3600):

| | | |
|---|---|---|
| T4 DNA Ligase 2 x Buffer | 5 | μl |
| pGEM-T Vector (50 ng) | 1 | μl |
| PCR product | 50 | ng |
| T4 DNA Ligase (3 Weiss units/μl) | 1 | μl |
| dH₂O to a final volume of | 10 | μl |

Conditions:

Incubate the reactions overnights at 4° C.

Then we transformed 2-4 μl ligation products into 50 μl JM109 high efficiency competent cells by the “heat shock” method (J. Sambrook, E. F. Fritsch, T. Maniatis (1989) Molecular Cloning 1.74, 1.84). Transformation cultures were plated onto the LB plates with ampicillin/IPTG/X-Gal, and these plates were incubated overnight at 37° C. Recombinant clones were identified by colour screening on indicator plates and colony PCR screening. The positive clones were inoculated into 3 ml LB liquid medium and incubated overnight at 37° C. with shaking (˜250 rpm). The cells were sedimented by centrifugation for 5 min at 10,000×g, and a plasmid sample was prepared from the cell pellet by using Minipreps DNA Purification System. Finally, the plasmids were sequenced with BigDye Terminator Cycle Sequencing Ready Reaction Kit by using ABI377 sequencer. The sequencing reaction was as follows:

| | | |
|---|---|---|
| Terminator Ready Reaction Mix | 8 | μl |
| Plasmid DNA | 1-1.5 | μg |
| Primer | 3.2 | pmol |
| dH₂O to a final volume of | 20 | μl |

The sequencing result showed that the PCR band obtained using primer 2, as well as primer 3 corresponds to the 3′end of the endoglucanase encoding sequence.

Cloning of 5′ End of the Endoglucanase

Based on the 3′-end sequence, we designed four specific primers which were used for 5′ end sequence cloning.

(SEQ ID NO: 8)

5′-1: 5′ AAG ATG TAC TGG GAA GTG 3′

(SEQ ID NO: 9)

5′-2: 5′ TGG TTG AGA TTG AGG ACT AAG 3′

(SEQ ID NO: 10)

5′-3: 5′ GAT TAT AGA ATT GTA GTA TCT 3′

(SEQ ID NO: 11)

5′-4: 5′ AGA GCC GGT CAT TGA GTT G 3′

The 5′RACE system was used to synthesize the 5′end fragment of the endoglucanase. 5 mg total RNA and primer 5′-1 was added for synthesis of the first strand. Then other primers were used for the second strand synthesis. The system and conditions of PCR of dC-tailed cDNA is as following:

| | | |
|---|---|---|
| 10x PCR buffer (200 mMTris-HCl(pH8.4), 500 mM KCl) | 5 | μl |
| 25 mM MgCl₂ | 3 | μl |
| 10 mM dNTP mix | 1 | μl |
| 5′Primer (10 μM) | 2 | μl |
| Abridged Anchor Primer(10 μM, provided by 3′RACE system) | 2 | μl |
| TaqDNA polymerase (5 u/μl) | 0.5 | μl |
| dC-tailed cDNA | 5 | μl |
| Add autoclaved, distilled water to | 50 | μl |

PCR Conditions:

| | | |
|---|---|---|
| 94° C. | 2 min | |
| 94° C. | 40 sec | |
| 53° C. | 40 sec | 30 cycles |
| 72° C. | 1 min | |
| 72° C. | 10 min | |

Two specific bands corresponding to approx. 700 bp and 400 bp resulted from using primers 5′-2, and 5′-4, respectively, using the 5′RACE system. The PCR-products were purified, ligated into the pGEM-T-vector, transformed into JM109 competent cells, and sequenced. The sequencing result showed we got the 5′end fragment of BG025.

Cloning of the Full Length Endoglucanase Gene

According to the above 3′ and 5′ end sequences, two primers for full length cloning were designed:

(SEQ ID NO: 12)

CDS-1: 5′ ATG AAG CTC GGC TCT CTC GT 3′

(SEQ ID NO: 13)

CDS-2: 5′ CTT GTC TCC TGT CTC GTT CAC 3′

Primer CDS-1 and AUAP was used for amplifying the full length gene from the cDNA. The following PCR reaction system and conditions were used:

| | |
|---|---|
| 10x PCR buffer | 5 μl |
| 25 mM MgCl₂ | 3 μl |
| 10 mM dNTP mix | 1 μl |
| Primer CDS-1 (10 μM) | 1 μl |
| AUAP (10 μM) | 1 μl |
| TaqDNA polymerase (5 u/μl) | 0.5 μl |
| cDNA synthesis reaction | 2 μl |
| Add autoclaved, distilled water to | 50 μl |

Conditions:

| | | |
|---|---|---|
| 95° C. | 2 min | |
| 95° C. | 40 sec | |
| 58° C. | 40 sec | 30 cycles |
| 72° C. | 1.5 min | |
| 72° C. | 10 min | |

From this amplification, a specific band with the size of about 1.2 kb was obtained and this was recovered from gel with the PCR Preps DNA Purification System. Then the purified fragment was ligated into the pGEM-T Vector, and transformed into the competent cells (JM109). Positive clones were screened by colony PCR, and the plasmid was extracted from these clones with Minipreps DNA Purification System. Finally the plasmid was sequenced with the BigDye Terminator Cycle Sequencing Ready Reaction Kit, and the full length endoglucanase encoding sequence was obtained.

### Example 6

### Determination of the Thermostability of Enzymes by Differential Scanning Calorimetry (DSC)

Endoglucanase

The purity of the purified endoglucanase resulting from Example 3 was determined by SDS-PAGE to be above 90%. The concentration of protein was determined to 1.9 mg/ml (based on OD₂₈₀ and an extinction coefficient calculated on the basis of the amino acid sequence).

For determining the denaturation or melting temperature of the endoglucanase (Td or Tm, respectively), the sample is dialysed over-night at 4° C. against a buffer containing 10 mM sodium phosphate, 50 mM sodium chloride, pH7.0. The dialysed sample was measured against pure buffer in a Microcalorimeter (VP-DSC from Microcal) from 20° C. to 95-100° C. with a temperature gradient of 1.5° C./min. The melting temperature was determined as the summit of the peak in the resulting thermogram: Tm 77.5° C. at about 0.0011 cal/deg.

Xylanase

A sample of the xylanase derived from Thermomyces lanuginosus (see Examples 1-3 of WO 96/23062) of a purity of above 90% as determined by SDS-PAGE, and a concentration of protein of 0.8 mg/ml (based on OD₂₈₀ and an extinction coefficient calculated on the basis of the amino acid sequence) was subjected to a procedure as described above, and Tm was determined to 75.0° C. at about −0.0008 cal/deg.

### Example 7

### Pelleting Stability of Enzyme Compositions

In this example, various enzyme compositions were added to feed and subjected to pelleting experiments at typical industry conditions (75° C.), as well as at aggressive processing conditions (85° C.). The recovery of each enzyme was determined. The temperatures (75° C., 85° C.) refer to the temperature of feed samples at the outlet of the pelleting machine.

Feed Enzymes

The following enzymes formed part of enzyme compositions which were subjected to pelleting experiments:

| Enzyme Code | Enzyme Designation | Reference |
|---|---|---|
| Beta-glucanase A | Endoglucanase | Described herein. |
| | Cel5A of | |
| | Thermoascus | |
| | aurantiacus | |
| Beta-glucanase B | RONOZYME A | Enzyme preparation derived from Bacillus |
| | | amyloliquefaciens which contains beta- |
| | | glucanase (EC 3.2.1.6) and alpha-amylase. |
| | | (EC 3.2.1.1). Commercially available from |
| | | Roche Vitamins AG, Switzerland |
| Beta-glucanase C | ROXAZYME G2 | Enzyme preparation derived from Trichoderma |
| | | longibrachiatum which contains cellulase, |
| | | endo-beta-1,3:4-glucanase, and xylanase. |
| | | Commercially available from Roche Vitamins |
| | | AG, Switzerland. |
| Beta-glucanase D | RONOZYME W | Enzyme preparation derived from Humicola |
| | | insolens which contains xylanase and beta- |
| | | glucanase. Commercially available from |
| | | Roche Vitamins AG, Switzerland. |
| Xylanase A | RONOZYME WX | Xylanase derived from Thermomyces |
| | | lanuginosus (described in WO 96/23062). |
| | | Commercially available from Roche Vitamins |
| | | AG, Switzerland. |
| Xylanase B | Humicola insolens | Xylanase described in EP 579672 |
| | xylanase 1 | |
| Xylanase C | ROXAZYME G2 | See above |
| Xylanase D | RONOZYME W | See above |
| Galactanase A | Myceliophthora | Galactanase described in WO 97/32014. |
| | thermophila | |
| | galactanase | |
| Galactanase B | Aspergillus | Galactanase described in WO 92/13945 |
| | aculeatus | |
| | galactanase | |
| Phytase A | Consensus Phytase | Consensus-phytase-10-thermo[3]-Q50T-K91A |
| | | described in WO 00/43503. |
| Phytase B | RONOZYME P | Phytase derived from Peniophora lycii |
| | | (described in WO 98/28408). Commercially |
| | | available from Roche Vitamins AG, |
| | | Switzerland. |

Pelletinq Experiments

Equipment: Mixer: TURBULA (lab-scale, up to 1-2 kg), FORBERG 60 V (pilot-scale mixer); Pelleting machine: BUHLER DFPL, nominal throughput 300 kg/h; Dryer: Cooling box with perforated bottom, ventilator.

Feed composition: Broiler MaisF4 with the following composition (%):

| | |
|---|---|
| Maize | 57.30 |
| Rice | 3.10 |
| Soya 50 | 28.60 |
| Fish meal | 3.00 |
| Soya oil | 2.00 |
| Starch | 2.00 |
| Lignosulfonate | 2.00 |
| Mineral Premix BV 4245 | 2.00 |
| Total | 100.00 |
| Size of used nozzle, die, | |
| mm | |
| for 75° C. | 3 × 30 |
| for 85° C. | 3 × 30 |

Additive premixes were prepared by spraying 300 g of each liquid enzyme sample to be tested, in a dilution providing application-relevant enzyme dosages according to the recommendations of the manufacturer, on top of 300 g of wheat middlings as a carrier, and mixing for 10 minutes using the TURBULA mixer. The additive premixes were then labelled and stored at cool temperature till use.

The additive premix (600 g) and the feed ingredients (29.4 kg) were added to the FORBERG mixer and mixed for around 2.5 minutes. The mash feed (30 kg) was then collected in paper bags (15 kg×2), labelled and stored at room temperature till further use.

Each of the two mash feed compositions (15 kg) were added to the pelleting machine for pelleting at either 75° C. or 85° C. It was conditioned with steam (125-130° C., pressure 1.0-1.2 bar) for around 10 seconds and then passed to the pelleting chamber where it was compacted. The pelleted feed was then transferred to the drier where it was ventilated with ambient air until ambient temperature was reached (around 6 minutes). The machine was run with a throughput level of around 35%, i.e. around 140 kg/h. The conditioning and pelleting temperatures were controlled by varying steam addition to target pelleting temperatures of 75° C. or 85° C., measured at the outlet of the press. The drying step was controlled so as to achieve a resulting moisture content of below 13%.

Samples of mash feed were taken in the mixer after mixing. For pelleted feed, sampling started from the product flow after around ⅔ of the time elapsed to produce an entire batch of feed (a batch of pelleted feed was made in around 5 minutes and sampling of around 5 kg was done around 3 minutes after start of production). The feed was poured on a plastic liner, quartered and three samples taken from the middle of the slices. The samples were packed in paper bags and labelled. The samples were stored protected from light at around 4° C. till assay.

The enzyme activity of mash feed and pellets was determined using the assays described below. Three samples of each batch were taken for each assay time point. Each sample was analysed twice and an average was calculated out of the six resulting values for mash and pelleted samples respectively.

Determination of Enzyme Activity

Beta-glucanase: Substrate: 1% AZO-beta-Glucan from barley (Megazyme Cat. No. S-ABG 100), incubation temperature 50° C. (Beta-glucanase B, C, D) or 65° C. (Beta-glucanase A), pH: 5.00. Extraction/Assay buffer: 50 g of a feed sample is extracted in 500 ml buffer, 45 min stirring (150 mM Na-phosphate buffer with 0.02% Tween 20 pH 5.0). Assay: 0.2 ml sample extract, 0.2 ml 1% AZO-beta-Glucan, mix and incubate 30-60 min. The reaction was stopped by adding 1.2 ml STOP-Reagent (40 g Na-acetate, 4 g zinc acetate add 150 ml dist. water and adjust pH with HCl conc. to pH 5.0 and fill up with dist. water to 200 ml. Add 800 ml 2-methoxy ethanol). After stopping the samples are mixed. After 15 min at room temperature the samples were centrifuged (3 min 15K rpm) and measured at 590 nm.

Xylanase: Substrate: 2% AZO-Xylan from birchwood (Megazyme Cat. No. S-AXBP) in 100 mM Na-phosphate buffer pH 5.0, incubation temperature: 50° C. (Xylanase B, C, D) or 65° C. (Xylanase A), pH: 5.00. Extraction/Assay buffer 50 g of a feed sample is extracted in 500 ml buffer, 45 min stirring (100 mM Na-phosphate buffer with 0.02% Tween 20 pH 5.0). Assay: 0.2 ml sample extract, 0.2 ml 1% AZO-Xylan, mix and incubate 30-120 min. The reaction was stopped by adding 1.2 ml STOP-Reagent (95% EtOH). After stopping the samples are mixed. After 15 min at room temperature the samples were centrifuged (3 min 15K rpm) and measured at 590 nm.

Galactanase: Mash and pellets were incubated (8 g/50 ml) for two hours using suitable pH and temperature conditions for each enzyme (ie extraction with water at 55° C. for Galactanase A and extraction with 0.2 M acetate buffer at pH 4.4 at 40° C. for Galactanase B). The samples were centrifuged and the amount of released galactose was determined using a commercial kit (Boehringer Mannheim Lactose/D-galactose kit). Briefly, D-galactose was oxidized at pH 8.6 by nicotinamide-adenine dinucleotide (AND+) to D-galactonic acid in the presence of the enzyme beta-galactose dehydrogenase (Gal-DH). The amount of NADH is directly stoichiometrically proportional to the amount of D-galactose (1 mol D-galactose results in 1 mol NADH). The increase in NADH is measured by means of its light absorbance at 340 nm.

Phytase: The phytase activity wass determined in the unit of FTU, one FTU being the amount of enzyme that liberates 1 micro-mol inorganic ortho-phosphate per min. under the following conditions: pH 5.5; temperature 37° C.; substrate: Sodium phytate (C₆H₆O₂₄P₆Na₁₂) in a concentration of 0.0050 mol/l (the FTU assay is described in Example 1 of WO 00/20569 (determination of phytase activity in feed and premix). Feed samples were extracted as described in WO 00/20569.

Calculations

% Recovery of enzymes in feed pelleted at either 75° C. and 85° C. is calculated relative to the activity of the mash sample:
% recovery=100*enzyme activity in feed pellets/enzyme activity in mash feed
   Results

The results are shown in Table 7 below, given as average and standard deviation of the activity units recovered in the pellets (n=3 samples taken at each of the pelleting temperatures) as well as the recovery (%) relative to the activity units in the mash.

TABLE 7

| | Pelleting at 75° C. | | | Pelleting at 85° C. | | |
|---|---|---|---|---|---|---|
| | Activity | | % | Activity | | % |
| | in | | Recov- | in | | Recov- |
| | pellets* | SD | ery** | pellets* | SD | ery** |
| Beta-glucanase | | | | | | |
| activity | | | | | | |
| Beta- | 473 | 5 | 96 | 452 | 5 | 91 |
| glucanase A | | | | | | |
| Beta- | 362 | 3 | 81 | 207 | 4 | 46 |
| glucanase B | | | | | | |
| Beta- | 293 | 4 | 58 | 125 | 3 | 25 |
| glucanase C | | | | | | |
| Beta- | 145 | 4 | 39 | ˜0 | — | 0 |
| glucanase D | | | | | | |
| Xylanase | | | | | | |
| activity | | | | | | |
| Xylanase A | 475 | 12 | 92 | 441 | 21 | 85 |
| Xylanase B | 248 | 33 | 75 | 224 | 14 | 68 |
| Xylanase C | 259 | 4 | 59 | 66 | 3 | 15 |
| Xylanase D | 231 | 4 | 86 | 80 | 4 | 30 |
| Galactanase | | | | | | |
| activity | | | | | | |
| Galactanase A | 3.51 | 0.1 | 91 | 2.38 | 0.03 | 62 |
| Galactanase B | 1.27 | 0.1 | 72 | 0.70 | 0.07 | 40 |
| Phytase activity | | | | | | |
| Phytase A | 1567 | 26 | 82 | 1479 | 33 | 77 |
| Phytase B | 1377 | 36 | 54 | 979 | 37 | 38 |

*Data given in activity units relevant for each analytical method
**Relative to the activity in the corresponding mash samples

### Deposit of Biological Material

The following biological material has been deposited under the terms of the Budapest Treaty with DSMZ (DSMZ-Deutsche Sammlung von Mikroorganismen und Zellkulturen GmbH, Mascheroder Weg 1b, D-38124 Braunschweig, Germany), and CGMCC (the China General Microbiological Culture Collection Center, Institute of Microbiology, Chinese Academy of Sciences, Haidian, Beijing 100080, China); and given the following accession

| Deposit | Accession Number | Date of Deposit |
|---|---|---|
| Escherichia coli | DSM 14541 | 2001-09-28 |
| Thermoascus aurantiacus | CGMCC No. 0670 | 2001-12-27 |

The deposits were made by Novozymes A/S, Krogshoejvej 36, DK-2880, Denmark, and Novozymes (China) Investment Co. Ltd., 22 Xinxi Zhong Lu, Shangdi zone, Haidian District, Beijing 100080, P.R. China, respectively, and the depositors have authorised the applicant to refer to this material and have given their unreserved and irrevocable consent to the deposited material being made available to the public in accordance with R. 28 EPC. The Escherichia coli strain harbours a plasmid containing the nucleic acid sequence of endoglucanase Cel5A of Thermoascus aurantiacus DSM 14541 (i.e. SEQ ID NO: 1 encoding SEQ ID NO:2).

These strains have been deposited under conditions that assure that access to the cultures will be available during the pendency of this patent application to one determined by the Commissioner of Patents and Trademarks to be entitled thereto under 37 C.F.R. §1.14 and 35 U.S.C. §122. The deposits represent a substantially pure culture of the deposited strains. The deposits are available as required by foreign patent laws in countries wherein counterparts of the present application, or its progeny are filed. However, it should be understood that the availability of a deposit does not constitute a license to practice the subject invention in derogation of patent rights granted by governmental action.

Thermoascus aurantiacus strain no. CGMCC 0670 was isolated from a soil sample collected on Jul. 21, 1998 in the Yunnan Province, Xishuangbanna, China.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

| | |
|---|---|
| Form - PCT/RO/134 (EASY) | |
| Indications Relating to Deposited | |
| Microorganism(s) or Other Biological | |
| Material (PCT Rule 13bis) | |
| Prepared using | PCT-EASY Version 2.92 |
| | (updated 01.01.2003) |
| International Application No. | |
| Applicant's or agent's file reference | 10254.204-WO |
| The indications made below relate to | |
| the deposited microorganism(s) or | |
| other biological material referred to | |
| in the description on: | |
| page | 50 |
| line | 3 |
| Identification of Deposit | |
| Name of depositary institution | DSMZ-Deutsche Sammlung von |
| | Mikroorganismen und |
| | Zellkulturen GmbH |
| Address of depositary institution | Mascheroder Weg 1b, D-38124 |
| | Braunschweig, Germany |
| Date of deposlt | 28 September 2001 (28.09.2001) |
| Accession Number | DSMZ 14541 |
| Additional Indications | NONE |
| Designated States for Which | all designated States |
| Indications are Made | |
| Separate Furnishing of Indications | NONE |
| These indications will be submitted to | |
| the International Bureau later | |
| The indications made below relate to | |
| the deposited microorganism(s) or | |
| other biological material referred to | |
| in the description on: | |
| page | 50 |
| line | 4 |
| Identification of Deposit | |
| Name of depositary institution | China General Microbiological |
| | Culture Collection Center |
| Address of depositary institution | China Committee for Culture |
| | Collection of Microorganisms, |
| | P.O. Box 2714, Beijing 100080, |
| | China |
| Date of deposit | 27 December 2001 (27.12.2001) |
| Accession Number | CGMCC 0670 |
| Additional indications | NONE |
| Designated States for Which | all designated States |
| Indications are Made | |
| Separate Furnishing of Indications | NONE |
| These indications will be submitted to | |
| the International Bureau later | |

1-13. (canceled)

14. A composition comprising at least two thermostable enzymes selected from the group consisting of endoglucanase, xylanase, phytase, protease, galactanase, mannanase, dextranase, and alpha-galactosidase, wherein each of the thermostable enzymes has a melting temperature, Tm, of at least 70° C., as determined by Differential Scanning Calorimetry (DSC) at a pH in the interval of 5.0 to 7.0.

15. The composition of claim 14, which comprises the following thermostable enzymes: (i) endoglucanase and xylanase; (ii) endoglucanase and protease; (iii) endoglucanase, xylanase and phytase; (iv) endoglucanase, xylanase and protease; (v) endoglucanase, xylanase, phytase and protease; (vi) xylanase and phytase; (vii) xylanase and protease; (viii) phytase and protease; (ix) phytase, protease and galactanase; (x) xylanase, phytase and protease; (xi) xylanase, protease and galactanase; (xii) phytase and galactanase; (xiii) galactanase and protease; (xiv) phytase, galactanase and alpha-galactosidase; (xv) phytase and alpha-galactosidase; (xvi) protease and alpha-galactosidase; (xvii) galactanase and alpha-galactosidase; (xviii) galactanase, protease and alpha-galactosidase; or (xix) at least two of endoglucanase, xylanase, phytase and galactanase.

16. The composition of claim 14, which comprises
(A) at least one polypeptide having xylanase activity, which is a family 11 glycoside hydrolase; and
(B) at least one polypeptide having endoglucanase activity, which
(i) has an amino acid sequence of at least 75% identity to amino acids −30 to 305 or 1 to 305 of SEQ ID NO:2 or 1 to 303 of SEQ ID NO: 18,
(ii) is encoded by a nucleic acid sequence which hybridizes under low stringency conditions with
(a) the mature endoglucanase encoding part of the plasmid contained in Escherichia coli DSM 14541,
(b) nucleotides 1 to 1008 or 91 to 1008 of SEQ ID NO:1 or 97 to 1008 of SEQ ID NO: 17,
(c) a subsequence of (a) or (b) of at least 100 nucleotides, or
(d) a complementary strand of (a), (b) or (c);
(iii) is a fragment of (i) or (ii) that has endoglucanase activity.

17. The composition of claim 16, wherein
(a) the endoglucanase and the xylanase are thermostable;
(b) the polypeptide having endoglucanase activity is a family 5 glycoside hydrolase; and/or
(c) the polypeptide having xylanase activity is derived from a strain of Aspergillus, Bacillus, Humicola, Thermomyces, or Trichoderma.

18. The composition of claim 14, further comprising
(a) at least one fat soluble vitamin, and/or
(b) at least one water soluble vitamin, and/or
(c) at least one trace mineral.

19. The composition of claim 14 which is an animal feed additive.

20. An animal feed composition having a crude protein content of 50 to 800 g/kg and comprising the composition of claim 14.

21. The animal feed composition of claim 20, which comprises at least one of soy, wheat, barley, oats or rye.

22. A method for the treatment of vegetable proteins, comprising the step of adding the composition of claim 14 to at least one vegetable protein or protein source.

23. The method of claim 22, wherein the vegetable protein source comprises at least one of soy, wheat, barley, oats and rye.

24. A method for improving the nutritional value of an animal feed, comprising adding the composition of claim 14 to the feed.
